(19) United States
(12) Patent Application Publication
(10) Pub. No.: US 2025/0272950 A1
(43) Pub. Date: Aug. 28, 2025

(54) TEST CHART, IMAGE ANALYSIS APPARATUS, IMAGE ANALYSIS SYSTEM, IMAGE ANALYSIS METHOD, AND NON-TRANSITORY COMPUTER READABLE MEDIUM

(71) Applicant: NEC Corporation, Minato-ku, Tokyo (JP)

(72) Inventor: Chisato Funayama, Tokyo (JP)

(73) Assignee: NEC Corporation, Minato-ku, Tokyo (JP)

(21) Appl. No.: 18/857,224

(22) PCT Filed: Jul. 26, 2022
(86) PCT No.: PCT/JP2022/028796

§ 371 Date: Oct. 16, 2024

(51) Int. Cl. G06V 10/60; G06T 5/50; G06T 7/50; G06V 10/143; H04N 23/21

(52) U.S. Cl. CPC G06V 10/60; G06T 5/50; G06T 7/50; G06V 10/143; H04N 23/21; G06T 2207/20221

(57) ABSTRACT

A test chart TC1 includes a sheet member ST, and a plurality of first skin reflection regions SA1 to SA4 formed on the sheet member ST. Each of the plurality of first skin reflection regions SA1 to SA4 indicates a reflectance of a skin with respect to first wavelength light.

### TECHNICAL FIELD

The present disclosure relates to a test chart, an image analysis apparatus, an image analysis system, an image analysis method, and a storage medium.

### BACKGROUND ART

For example, Patent Document 1 discloses a display method of a digital color face image that photographs a face of a target person, applies cosmetic treatment or hair makeup treatment on an image of the photographed face by image processing, and displays the face image on a display unit.

In the display method described in Patent Document 1, at a time of photographing by a camera, a color chart whose colorimetric value luminance (reflectance) is known is photographed in the same environment as an environment in which a face image is photographed. An illuminance correction factor Q1 in a photographing environment is computed from an RGB value of a color chart image output from the camera to a computer apparatus, the photographed face image is introduced to the computer apparatus, and illuminance correction is performed by converting an RGB component of each pixel of the entirety of the image into a conversion output level to a colorimetric value luminance with use of the illuminance correction factor Q1. Correction is performed into an output level by the camera with respect to a face image that has undergone conversion into the conversion output level to the colorimetric value luminance with use of the illuminance correction factor Q1 in the photographing environment by using a pre-adjusted illuminance correction factor Q2 that “achieves brightness with which a face (skin color) looks preferable”, and the corrected face image is output to a display.

Patent Documents 2 and 3 disclose a test chart of a gray scale.

Patent Document 2 discloses a test chart in which light from a light emitting portion is transmitted. The test chart integrally includes gray scale portions 23A and 23B in which a plurality of portions whose light transmittance is stepwise different are arranged in parallel, and a resolution chart portion 24 composed of light and dark stripes of a predetermined pitch.

Patent Document 3 discloses a test chart in which two patterns being a same gradation pattern are arranged point symmetric with respect to a center point of a plane.

### Related Document

### Patent Document

Patent Document 1: Japanese Patent Application Publication No. 2002-10283
Patent Document 2: Japanese Patent Application Publication No. H07-222206
Patent Document 3: Japanese Patent Application Publication No. 2001-268363

### SUMMARY

### Technical Problem

An object of the present disclosure is to improve the techniques described in the above-described related documents.

### Solution to Problem

One aspect of the present disclosure provides a test chart including:
a sheet member; and
a plurality of first skin reflection regions formed on the sheet member, wherein
each of the plurality of first skin reflection regions indicates a reflectance of a skin with respect to first wavelength light.

One aspect of the present disclosure provides an image analysis apparatus including:
an image acquisition unit that acquires a chart image to be acquired by photographing a test chart; and
an analysis unit that analyzes the chart image, wherein
the test chart includes
a sheet member, and
a plurality of first skin reflection regions formed on the sheet member, and
each of the plurality of first skin reflection regions indicates a reflectance of a skin with respect to first wavelength light.

One aspect of the present disclosure provides an image analysis system including:
a test chart;
a camera that photographs the test chart and generates a chart image;
a first light that irradiates the first wavelength light to the test chart; and
an image analysis apparatus, wherein
the test chart includes
a sheet member, and
a plurality of first skin reflection regions formed on the sheet member,
each of the plurality of first skin reflection regions indicates a reflectance of a skin with respect to first wavelength light, and
the image analysis apparatus includes
an image acquisition unit that acquires a chart image to be acquired by photographing the test chart, and
an analysis unit that analyzes the chart image.

One aspect of the present disclosure provides an image analysis method including, by one or more computers:
acquiring a chart image to be acquired by photographing a test chart; and
analyzing the chart image, wherein
the test chart includes
a sheet member, and
a plurality of first skin reflection regions formed on the sheet member, and
each of the plurality of first skin reflection regions indicates a reflectance of a skin with respect to first wavelength light.

One aspect of the present disclosure provides a storage medium storing a program for causing a computer to execute:
acquiring a chart image to be acquired by photographing a test chart; and
analyzing the chart image, wherein
the test chart includes
a sheet member, and
a plurality of first skin reflection regions formed on the sheet member, and
each of the plurality of first skin reflection regions indicates a reflectance of a skin with respect to first wavelength light.

### BRIEF DESCRIPTION OF THE DRAWINGS

FIG. 1 is a diagram illustrating a test chart according to an example embodiment 1.

FIG. 2 is a diagram illustrating a configuration example of an image analysis system according to the example embodiment 1.

FIG. 3 is a diagram illustrating an overview of an image analysis apparatus according to the example embodiment 1.

FIG. 4 is a flowchart illustrating an overview of an image analysis method according to the example embodiment 1.

FIG. 5 is a diagram illustrating a detailed example of a functional configuration of the image analysis apparatus according to the example embodiment 1.

FIG. 6 is a diagram illustrating a physical configuration example of the image analysis apparatus according to the example embodiment 1.

FIG. 7 is a flowchart illustrating a detailed example of the image analysis method according to the example embodiment 1.

FIG. 8 is a flowchart illustrating a detailed example of analysis processing according to the example embodiment 1.

FIG. 9 is a diagram illustrating one example of a display image in a case where it is decided that all first skin reflection images SAim1 to SAim4 are a good image.

FIG. 10 is a diagram illustrating one example of a display image in a case where it is decided that only the first skin reflection image SAim1 is not a good image.

FIG. 11 is a diagram illustrating one example of a display image in a case where it is decided that only the first skin reflection image SAim3 and the first skin reflection image SAim4 are not a good image.

FIG. 12 is a diagram illustrating one example of a display image in a case where it is decided that all the first skin reflection images SAim1 to SAim4 are not a good image.

FIG. 13 is a diagram illustrating a test chart according to an example embodiment 2.

FIG. 14 is a diagram illustrating a configuration example of an image analysis system according to the example embodiment 2.

FIG. 15 is a diagram illustrating one example of a display image in a case where it is decided that all second skin reflection images SBim1 to SBim4 are a good image.

FIG. 16 is a diagram illustrating one example of a display image in a case where it is decided that only the second skin reflection image SBim1 is not a good image.

FIG. 17 is a diagram illustrating one example of a display image in a case where it is decided that only the second skin reflection image SBim3 and the second skin reflection image SBim4 are not a good image.

FIG. 18 is a diagram illustrating one example of a display image in a case where it is decided that all the second skin reflection images SBim1 to SBim4 are not a good image.

FIG. 19 is a diagram illustrating a configuration example of an image analysis system according to an example embodiment 3.

FIG. 20 is a diagram illustrating a functional configuration example of an image analysis apparatus according to the example embodiment 3.

FIG. 21 is a flowchart illustrating one example of an image analysis method according to the example embodiment 3.

FIG. 22 is a diagram illustrating a configuration example of an image analysis system according to an example embodiment 4.

FIG. 23 is a diagram illustrating a functional configuration example of an image analysis apparatus according to the example embodiment 4.

FIG. 24 is a flowchart illustrating one example of an image analysis method according to the example embodiment 4.

FIG. 25 is a flowchart illustrating a detailed example of analysis processing according to the example embodiment 4.

FIG. 26 is a diagram illustrating a first example of a first image region and a second image region in a case where a region determination condition associated with a position where a first light is installed is set.

FIG. 27 is a diagram illustrating a second example of a first image region and a second image region in a case where a region determination condition associated with a position where the first light is installed is set.

FIG. 28 is a diagram illustrating a configuration example of a test chart according to an example embodiment 5.

FIG. 29 is a diagram illustrating a configuration example of an image analysis system according to the example embodiment 5.

FIG. 30 is a diagram illustrating a functional configuration example of an image analysis apparatus according to the example embodiment 5.

FIG. 31 is a flowchart illustrating one example of an image analysis method according to the example embodiment 5.

FIG. 32 is a flowchart illustrating a detailed example of analysis processing according to the example embodiment 5.

FIG. 33 is a diagram illustrating a third example of a first image region and a second image region in a case where a region determination condition associated with a position where a first light is installed is set.

### EXAMPLE EMBODIMENT

Hereinafter, example embodiments according to the present disclosure are described by using the drawings. Note that, in all drawings, a similar constituent element is indicated by a similar reference sign, and description thereof is omitted as necessary.

### Example Embodiment 1

### (Overview)

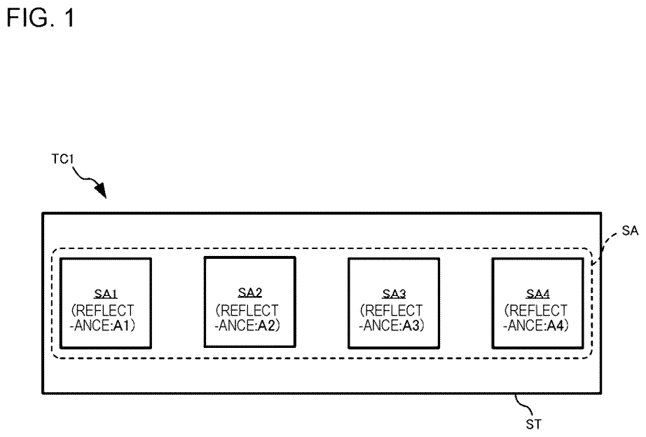

FIG. 1 is a diagram illustrating a test chart TC1 according to an example embodiment 1. The test chart TC1 includes a sheet member ST, and a plurality of first skin reflection regions SA1 to SA4 formed on the sheet member ST. Each of the plurality of first skin reflection regions SA1 to SA4 indicates a reflectance of a skin with respect to first wavelength light.

The test chart TC1 enables to improve the techniques described in the above-described related documents.

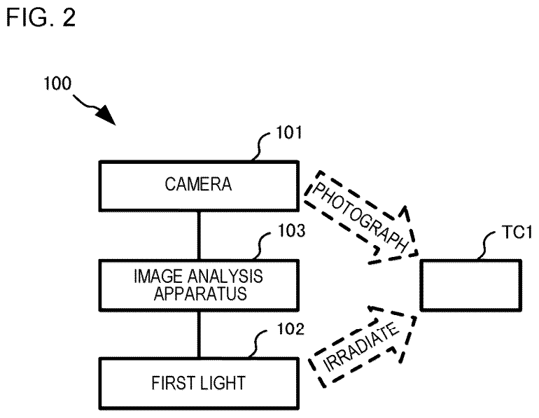

FIG. 2 is a diagram illustrating a configuration example of an image analysis system 100 according to the example embodiment 1. The image analysis system 100 includes the test chart TC1, a camera 101, a first light 102, and an image analysis apparatus 103.

The camera 101 photographs the test chart TC1, and generates a chart image. The first light 102 irradiates the first wavelength light to the test chart TC1.

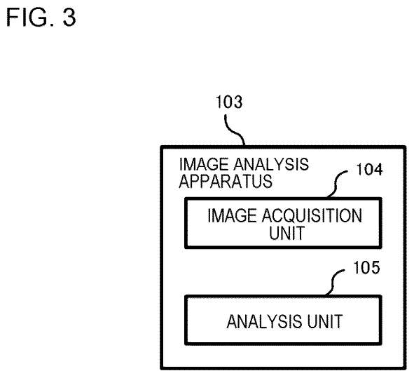

FIG. 3 is a diagram illustrating an overview of the image analysis apparatus 103 according to the example embodiment 1.

The image analysis apparatus 103 includes an image acquisition unit 104 that acquires a chart image to be acquired by photographing the test chart TC1, and an analysis unit 105 that analyzes the chart image.

The image analysis system 100 enables to acquire an appropriate skin image by photographing using light of a specific wavelength. Further, the image analysis apparatus 103 enables to improve the techniques described in the above-described related documents.

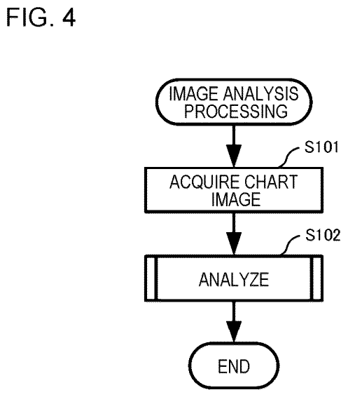

FIG. 4 is a flowchart illustrating an overview of an image analysis method according to the example embodiment 1.

The image acquisition unit 104 acquires a chart image to be acquired by photographing the test chart TC1 (step S101). The analysis unit 105 analyzes the chart image (step S102).

The image analysis method enables to improve the techniques described in the above-described related documents.

Hereinafter, a detailed example of the test chart TC1, the image analysis system 100, the image analysis apparatus 103, and the image analysis method according to the example embodiment 1 is described.

### (Details)

Generally, in an image for use in face authentication and the like, it is often a case that light of a specific wavelength for photographing is irradiated to a subject in a case where photographing is performed. Further, generally, a reflectance of a skin of a person with respect to light of a specific wavelength varies according to a state (e.g., a physical condition, makeup, and the like) of the person, an innate trait, and the like. Therefore, in an image in which various persons are photographed by using light of a specific wavelength, overexposure, black crushing, and the like may occur according to a reflectance of a skin with respect to the light, and thereby a low quality image may be generated.

For example, in the technique described in Patent Document 1, as described above, an image is corrected by using the illuminance correction factor Q1 and the illuminance correction factor Q2. In a case where an image (skin image) itself indicating a skin in an image generated by photographing is a low quality image including overexposure, black crushing, and the like, even when the technique described in Patent Document 1 is used, it may be difficult to acquire an appropriate skin image by photographing using light of a specific wavelength.

Further, for example, in the techniques described in Patent Documents 2 and 3, even when it is possible to acquire an image in which a gradation pattern is appropriately photographed, it may be difficult to acquire an appropriate skin image by photographing using light of a specific wavelength.

In view of a circumstance as described above, one example of an object of the present disclosure is to provide a test chart, an image analysis apparatus, an image analysis system, an image analysis method, and a storage medium that solve a task of acquiring an appropriate skin image by photographing using light of a specific wavelength.

### (Configuration Example of Test Chart TC1)

The test chart TC1 is a tool for checking in advance a light environment in a place (photographing place) where photographing of a person is performed for face authentication and the like. A light environment is an environment (e.g., a distribution condition of light) related to light, and light that produces a light environment may be various types of light such as natural light and illumination light. Natural light is, for example, sunlight. Illumination light is light for use in illumination, and a light source thereof may be, for example, a light-emitting diode (LED), a fluorescent lamp, an incandescent lamp, or the like.

As illustrated in FIG. 1, the test chart TC1 includes the sheet member ST, and a first region SA being appropriately set on one principal plane of the sheet member ST.

The sheet member ST is, for example, a rectangular sheet of about 200 millimeters (mm) in width, and about 100 to 300 mm in length.

The sheet member ST is desirably non-translucent, and a material thereof is, for example, cardboard, plastic, wood, metal, glass, or combination of one or a plurality thereof. As far as the material of the sheet member ST is cardboard, plastic, wood, or the like, the sheet member ST becomes lightweight, and thereby it makes easy to carry the sheet member ST.

Note that, a shape, a size, and the material of the sheet member ST are not limited to the above, and may be changed appropriately:

The plurality of first skin reflection regions SA1 to SA4 are formed in the first region SA. Each of the plurality of first skin reflection regions SA1 to SA4 indicates a reflectance (unit is, for example, %) of a skin with respect to the first wavelength light. Each of the first skin reflection regions SA1 to SA4 is also referred to as a color chart, a patch, or the like. Note that, a plurality of (specifically, two or more) first skin reflection regions may be formed in the first region SA, and the number of the first skin reflection regions is not limited to four.

The first wavelength light is light for use in photographing of a subject. A wavelength of the first wavelength light is a first wavelength. For example, the first wavelength light is near infrared light, and the first wavelength in this case is a specific wavelength to be selected from among 780 to 2500 nm.

In an example illustrated in FIG. 1, the first skin reflection region SA1 is a region associated with a skin of a person whose reflectance with respect to the first wavelength light is A1. The first skin reflection region SA2 is a region associated with a skin of a person whose reflectance with respect to the first wavelength light is A2. The first skin reflection region SA3 is a region associated with a skin of a person whose reflectance with respect to the first wavelength light is A3. The first skin reflection region SA4 is a region associated with a skin of a person whose reflectance with respect to the first wavelength light is A4.

The reflectances A1 to A4 differ from one another. A magnitude of A1 to A4 according to the present example embodiment is A1>A2>A3>A4.

A1 is a maximum reflectance of a skin with respect to the first wavelength light. Specifically, the first skin reflection region SA1 is equivalent to a “first maximum region” indicating a maximum reflectance of a skin with respect to the first wavelength light.

A4 is a minimum reflectance of a skin with respect to the first wavelength light. Specifically, the first skin reflection region SA4 is equivalent to a “first minimum region” indicating a minimum reflectance of a skin with respect to the first wavelength light.

Herein, generally, in an environment in which light with a specific wavelength is irradiated to a skin of a person, a reflectance of a skin of a person becomes a value between a maximum reflectance and a minimum reflectance according to a wavelength of light to be irradiated. For example, in a case where light to be irradiated is near infrared light, a maximum reflectance is about 70 to 95(%), and a minimum reflectance is about 5 to 30(%). A maximum reflectance and a minimum reflectance of a skin of a person with respect to light (e.g., first wavelength light) with a specific wavelength as described above can be acquired, for example, by irradiating light with the specific wavelength to a large number of persons, and measuring a reflectance with respect to the light.

Note that, the plurality of first skin reflection regions SA1 to SA4 may include the first maximum region and the first minimum region, and the number of the first skin reflection regions may be changed appropriately, as far as the number is two or more.

There are various manufacturing methods of the test chart TC1, and one example of the methods is described in the following.

First, a worker prepares a plurality of pieces of paper on which each of the plurality of first skin reflection regions SA1 to SA4 is formed, and pastes the each piece of paper to a mount. The plurality of pieces of paper are, for example, paper on which paint whose reflectance is adjusted to a reflectance of each of the first skin reflection regions SA1 to SA4 is coated. The paint is, for example, matte lacquer-based paint, but is not limited thereto.

Next, the worker prepares black cardboard paper of the same size as a size of the mount, and cuts out a portion of the cardboard paper associated with the plurality of pieces of paper (specifically, the plurality of first skin reflection regions SA1 to SA4) pasted to the mount. Then, the worker pastes together the black cardboard paper, and the mount to which the plurality of pieces of paper are pasted. The black cardboard paper desirably has a reflectance of 0 (zero), for example, with respect to near infrared light. This enables to manufacture the test chart TC1. In this example, the sheet member ST is composed of a mount and black cardboard paper.

### (Configuration Example of Image Analysis System 100)

The camera 101 is a camera for photographing a subject for face authentication and the like. In a case where an image acquired by photographing is used in face authentication, the camera 101 is provided in such a way as to photograph a face of a person. The camera 101 photographs the test chart TC1 in advance preparation before photographing a subject, and generates a chart image. Note that, the camera 101 may be a near infrared camera capable of photographing an iris image for iris authentication.

The first light 102 is a light for irradiating the first wavelength light to a subject. In a case where an image acquired by photographing is used in face authentication, the first light 102 is provided, for example, in such a way as to irradiate the first wavelength light to a face of a person during a time when the camera 101 performs photographing. The first light 102 may irradiate the first wavelength light to the test chart TC1 in advance preparation before the camera 101 photographs a subject.

Note that, the image analysis system 100 may include a plurality of first lights 102.

### (Functional Configuration Example of Image Analysis Apparatus 103)

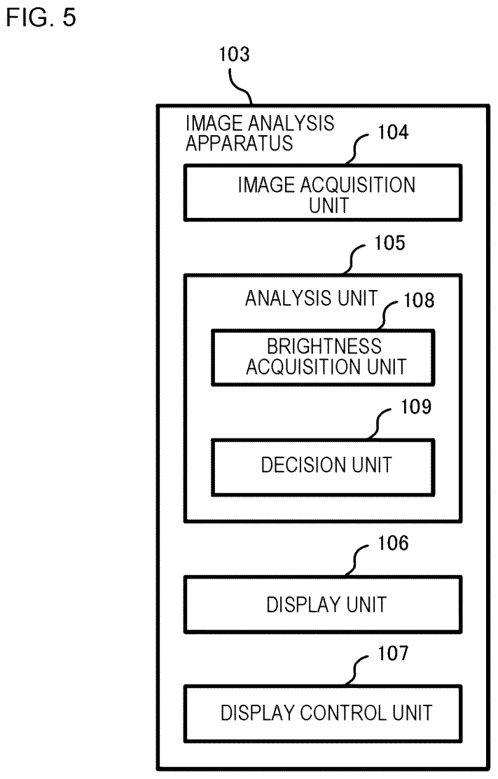

FIG. 5 is a diagram illustrating a detailed example of a functional configuration of the image analysis apparatus 103 according to the example embodiment 1. The image analysis apparatus 103 is an apparatus that analyzes a chart image to be generated by photographing the test chart TC1.

The image analysis apparatus 103 functionally includes, for example, the image acquisition unit 104, the analysis unit 105, a display unit 106, and a display control unit 107.

The image acquisition unit 104 acquires, for example, a chart image generated by the camera 101. The image acquisition unit 104 acquires from the camera 101 via a communication line configured, for example, by wired connection, wireless connection, or combining these.

The analysis unit 105 analyzes a chart image. The analysis unit 105 functionally includes a brightness acquisition unit 108, and a decision unit 109.

The brightness acquisition unit 108 derives brightness of each of a plurality of first skin reflection images SAim1 to SAim4. The first skin reflection images SAim1 to SAim4 are image portions each indicating the plurality of first skin reflection regions SA1 to SA4 in a chart image.

The decision unit 109 decides whether each of the plurality of first skin reflection images SAim1 to SAim4 is a good image that satisfies a predetermined first decision criterion, based on brightness of each of the plurality of first skin reflection images SAim1 to SAim4.

The first decision criterion is, for example, that brightness of the first skin reflection images SAim1 to SAim4 is less than a first threshold value, and more than a second threshold value. The first threshold value is a value more than the second threshold value.

The first threshold value is a value to be appropriately set for deciding whether overexposure has occurred. Specifically, the decision unit 109 decides whether overexposure has occurred in each of the first skin reflection images SAim1 to SAim4 by comparing brightness with the first threshold value.

The second threshold value is a value to be appropriately set for deciding whether black crushing has occurred. Specifically, the decision unit 109 decides whether black crushing has occurred in each of the first skin reflection images SAim1 to SAim4 by comparing brightness with the second threshold value.

In this case, for example, the decision unit 109 decides that the first skin reflection images SAim1 to SAim4 in which both of overexposure and black crushing have not occurred are a good image. The decision unit 109 decides that the first skin reflection images SAim1 to SAim4 in which overexposure or black crushing has occurred are not a good image.

The display unit 106 displays various pieces of information under control of the display control unit 107. The display control unit 107 causes the display unit 106 to display, for example, an analysis result of a chart image. Specifically, for example, the display control unit 107 generates a display image in which an analysis result of a chart image is superimposed on the chart image, and causes the display unit 106 to display the generated display image.

### (Physical Configuration Example of Image Analysis Apparatus 103)

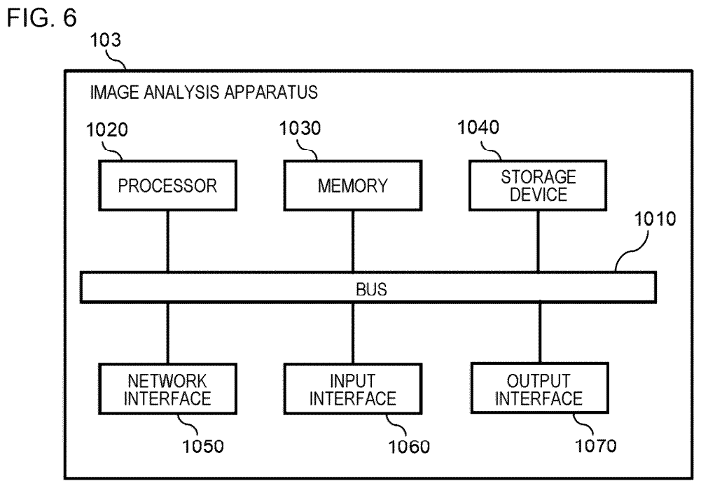

FIG. 6 is a diagram illustrating a physical configuration example of the image analysis apparatus 103 according to the example embodiment 1. The image analysis apparatus 103 is, for example, configured of a computer.

The image analysis apparatus 103 includes, for example, a bus 1010, a processor 1020, a memory 1030, a storage device 1040, a network interface 1050, an input interface 1060, and an output interface 1070.

The bus 1010 is a data transmission path along which the processor 1020, the memory 1030, the storage device 1040, the network interface 1050, the input interface 1060, and the output interface 1070 mutually transmit and receive data. However, a method of mutually connecting the processor 1020 and the like is not limited to bus connection.

The processor 1020 is a processor to be achieved by a central processing unit (CPU), a graphics processing unit (GPU), or the like.

The memory 1030 is a main storage apparatus to be achieved by a random access memory (RAM) or the like.

The storage device 1040 is an auxiliary storage apparatus to be achieved by a hard disk drive (HDD), a solid state drive (SSD), a memory card, a read only memory (ROM), or the like. The storage device 1040 stores a program module for achieving each function of the image analysis apparatus 103. Each function associated with a program module is achieved by causing the processor 1020 to read each program module in the memory 1030 and execute the read program module.

The network interface 1050 is an interface for connecting the image analysis apparatus 103 to a communication line.

The input interface 1060 is, for example, an interface for allowing a user to input information, and is, for example, configured of a touch panel, a keyboard, a mouse, and the like.

The output interface 1070 is an interface for presenting information to a user, and is, for example, configured of a liquid crystal panel, an organic electro-luminescence (EL) panel, or the like.

Note that, a physical configuration of the image analysis apparatus 103 is not limited to the one described herein, and may be changed appropriately. The image analysis apparatus 103 may be, for example, configured of a plurality of computers. Further, for example, one or both of the camera 101 and the first light 102 may be integrally configured with the image analysis apparatus 103.

### (Detailed Example of Image Analysis Method According to Example Embodiment 1)

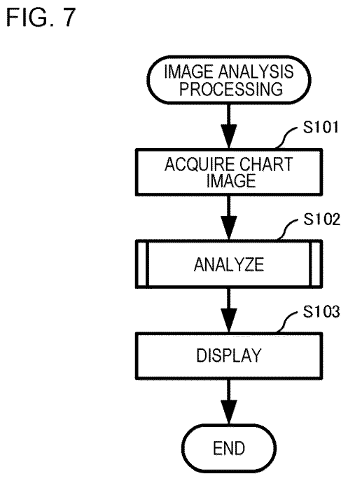

FIG. 7 is a flowchart illustrating a detailed example of an image analysis method according to the example embodiment 1. The image analysis method is a method for analyzing a chart image to be generated by photographing the test chart TC1. The image analysis method is, for example, started as a trigger by photographing the test chart TC1 by the camera 101 in response to an instruction from a user. In a case where the photographing is performed, the test chart TC1 is installed, for example, at a place where a subject (e.g., a face of a person serving as an authentication target) is located in face authentication.

The image acquisition unit 104 acquires a chart image to be acquired by photographing the test chart TC1 (step S101).

Specifically, for example, the image acquisition unit 104 acquires a chart image from the camera 101.

The analysis unit 105 analyzes the chart image (step S102).

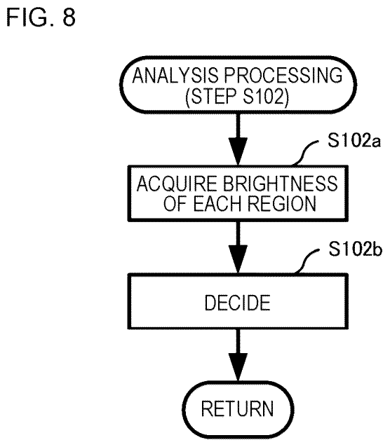

FIG. 8 is a flowchart illustrating a detailed example of analysis processing (step S102) according to the example embodiment 1.

The brightness acquisition unit 108 acquires brightness of each of the plurality of first skin reflection images SAim1 to SAim4, based on the chart image acquired in step S101 (step S102a).

Specifically, for example, the brightness acquisition unit 108 extracts a pixel value of each of the plurality of first skin reflection images SAim1 to SAim4 from the chart image acquired in step S101. The brightness acquisition unit 108 derives brightness of each of the plurality of first skin reflection images SAim1 to SAim4, based on the extracted pixel value.

The decision unit 109 decides whether each of the plurality of first skin reflection images SAim1 to SAim4 is a good image that satisfies the predetermined first decision criterion, based on the brightness acquired in step S102a (step S102b).

As described above, the first decision criterion is, for example, that brightness of the first skin reflection images SAim1 to SAim4 is less than the first threshold value, and more than the second threshold value.

Since overexposure has occurred in the first skin reflection images SAim1 to SAim4 in which brightness acquired in step S102a is equal to or more than the first threshold value, the decision unit 109 decides that the first skin reflection images SAim1 to SAim4 are not a good image (NG). Since black crushing has occurred in the first skin reflection images SAim1 to SAim4 in which brightness acquired in step S102a is equal to or less than the second threshold value, the decision unit 109 decides that the first skin reflection images SAim1 to SAim4 are not a good image (NG).

Neither overexposure nor black crushing has occurred in the first skin reflection images SAim1 to SAim4 in which brightness acquired in step S102a is less than the first threshold value, and more than the second threshold value. Therefore, the decision unit 109 decides that the first skin reflection images SAim1 to SAim4 as described above are a good image (OK).

After executing step S102b, the decision unit 109 returns to image analysis processing.

FIG. 7 is referred to again.

The display control unit 107 generates display images 111a to 111d, and causes the display unit 106 to display the generated display images 111a to 111d (step S103), and finishes the image analysis processing.

Specifically, for example, the display control unit 107 generates the display images 111a to 111d, based on the chart image chart image acquired in step S101, and an analysis result in step S102. For example, the display control unit 107 generates the display images 111a to 111d in which an analysis result in step S102 is superimposed on the chart image acquired in step S101.

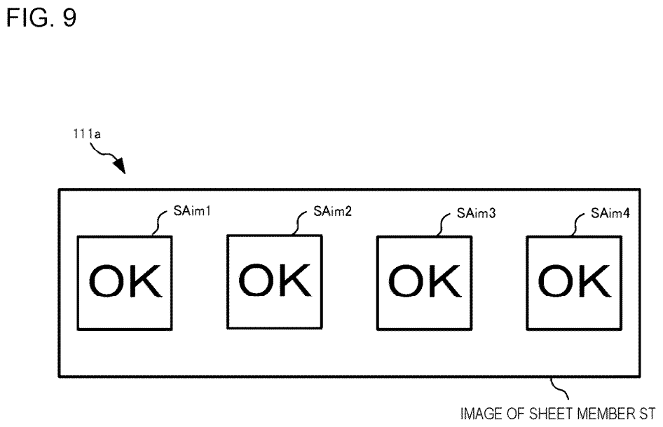

FIGS. 9 to 12 are diagrams illustrating an example of the display images 111a to 111d according to the present example embodiment.

FIG. 9 illustrates one example of the display image 111a in a case where all the first skin reflection images SAim1 to SAim4 are decided to be a good image. In this case, no matter what a value of a reflectance of a skin is, a possibility of being able to acquire a good image (e.g., a face image) to an extent that face authentication and the like can be performed is high. Therefore, it is clear that intensity of the first wavelength light is appropriate in a light environment in a photographing place.

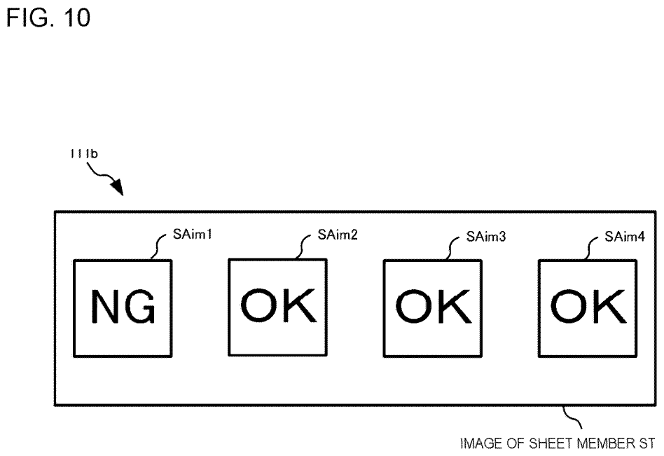

FIG. 10 illustrates one example of the display image 111b in a case where only the first skin reflection image SAim1 is decided not to be a good image. In this case, in a case of a skin whose reflectance with respect to the first wavelength light is large, there is a risk that a good image (e.g., a face image) to an extent that face authentication and the like can be performed for a person with the skin cannot be acquired. Therefore, it is clear that the first wavelength light may be too strong in a light environment in a photographing place.

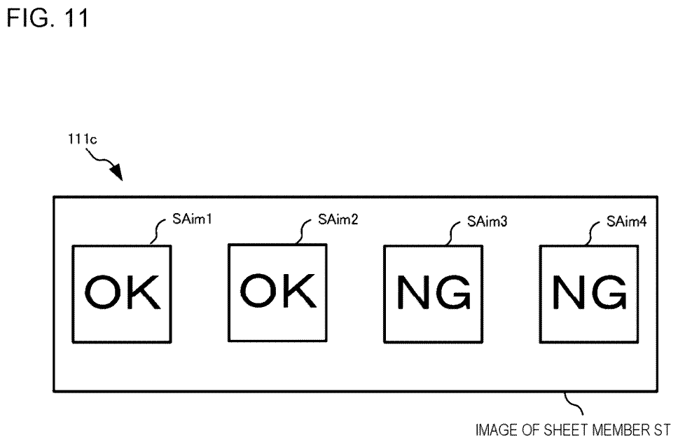

FIG. 11 illustrates one example of the display image 111c in a case where only the first skin reflection image SAim3 and the first skin reflection image SAim4 are decided not to be a good image. In this case, in a case of a skin whose reflectance with respect to the first wavelength light is small, there is a risk that a good image (e.g., a face image) to an extent that face authentication and the like can be performed for a person with the skin cannot be acquired. Therefore, it is clear that the first wavelength light may be weak in a light environment in a photographing place.

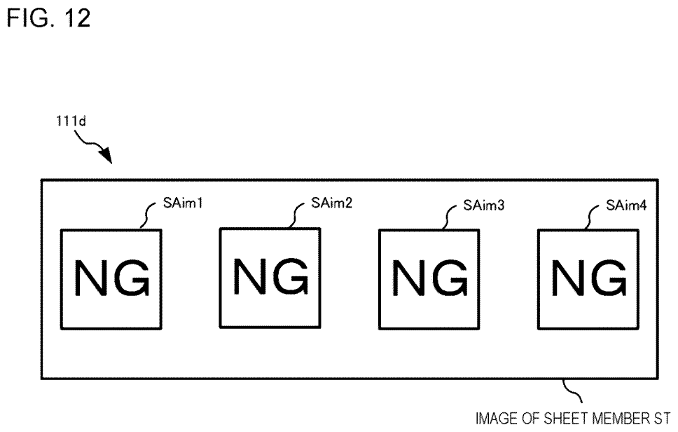

FIG. 12 illustrates one example of the display image 111d in a case where all the first skin reflection images SAim1 to SAim4 are decided not to be a good image. In this case, no matter what the value of a reflectance of a skin is, a possibility of not being able to acquire a good image (e.g., a face image) to an extent that face authentication and the like can be performed is high. Therefore, it is clear that the first wavelength light may be very weak in a light environment in a photographing place.

Performing the image analysis processing as described above enables to recognize whether a light environment in a photographing place is suitable for photographing for performing face authentication and the like, and handle in such a way that the light environment becomes appropriate in a case where the light environment is inappropriate. Therefore, it becomes possible to acquire an appropriate skin image by photographing using light of a specific wavelength. A skin image as described above can be used for biometric authentication such as face authentication and iris authentication, and in a case as described above, it becomes possible to acquire an appropriate image for biometric authentication, and consequently, more accurate biometric authentication is enabled.

### (Advantageous Effect)

As described above, according to the present example embodiment, the test chart TC1 includes the sheet member ST, and the plurality of first skin reflection regions SA1 to SA4 formed on the sheet member ST. Each of the plurality of first skin reflection regions SA1 to SA4 indicates a reflectance of a skin with respect to the first wavelength light.

This enables to check in advance whether to be able to acquire an appropriate image depending on an application, in a case where a subject is photographed by using the first wavelength light, and handle in such a way that a light environment becomes appropriate, in a case where the light environment is inappropriate. Therefore, it becomes possible to acquire an appropriate skin image by photographing using light of a specific wavelength.

According to the present example embodiment, the image analysis apparatus 103 includes the image acquisition unit 104 that acquires a chart image to be acquired by photographing the test chart TC1, and the analysis unit 105 that analyzes the chart image. The test chart TC1 includes the sheet member ST, and the plurality of first skin reflection regions SA1 to SA4 formed on the sheet member ST. Each of the plurality of first skin reflection regions SA1 to SA4 indicates a reflectance of a skin with respect to the first wavelength light.

This enables to check in advance whether to be able to acquire an appropriate image depending on an application, in a case where a subject is photographed by using the first wavelength light, and handle in such a way that a light environment becomes appropriate, in a case where the light environment is inappropriate. Therefore, it becomes possible to acquire an appropriate skin image by photographing using light of a specific wavelength.

According to the present example embodiment, the image analysis apparatus 103 further includes the display control unit 107. The display control unit 107 generates the display images 111a to 111d in which an analysis result of a chart image is superimposed on the chart image, and causes the display unit 106 to display the generated display images 111a to 111d.

This enables to easily recognize whether a light environment in a photographing place is an environment to be able to acquire an appropriate image depending on an application, in a case where a subject is photographed by using the first wavelength light. Therefore, it is possible to easily handle in such a way that a light environment becomes appropriate, in a case where the light environment is inappropriate. Therefore, it becomes possible to easily acquire an appropriate skin image by photographing using light of a specific wavelength.

According to the present example embodiment, the image analysis system 100 includes the test chart TC1, the camera 101, the first light 102, and the image analysis apparatus 103.

The test chart TC1 includes the sheet member ST, and the plurality of first skin reflection regions SA1 to SA4 formed on the sheet member ST. Each of the plurality of first skin reflection regions SA1 to SA4 indicates a reflectance of a skin with respect to the first wavelength light.

The camera 101 generates a chart image by photographing the test chart TC1. The first light 102 irradiates the first wavelength light to the test chart TC1.

The image analysis apparatus 103 includes the image acquisition unit 104 that acquires a chart image to be acquired by photographing the test chart TC1, and the analysis unit 105 that analyzes the chart image.

This enables to check in advance whether to be able to acquire an appropriate image depending on an application, in a case where a subject is photographed by using the first wavelength light, and handle in such a way that a light environment becomes appropriate, in a case where the light environment is inappropriate. Therefore, it becomes possible to acquire an appropriate skin image by photographing using light of a specific wavelength.

### Example Embodiment 2

In the present example embodiment, an example in which a test chart further includes a plurality of second skin reflection regions indicating a reflectance of a skin with respect to second wavelength light is described. In the present example embodiment, description overlapping the example embodiment 1 is omitted as necessary for simplifying description, and a point different from the example embodiment 1 is mainly described.

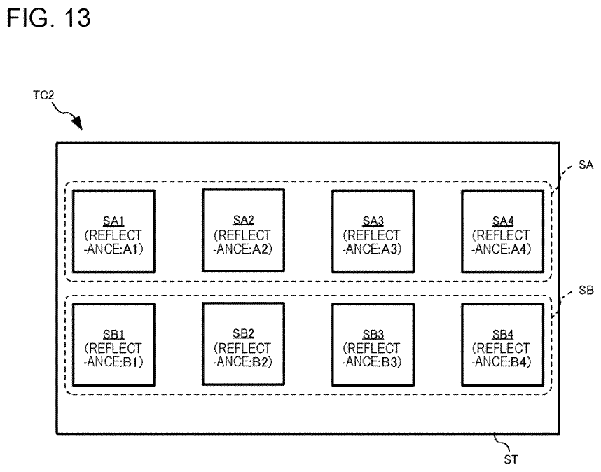

FIG. 13 is a diagram illustrating a test chart TC2 according to an example embodiment 2. The test chart TC2 includes, in addition to a sheet member ST and a plurality of first skin reflection regions SA1 to SA4 similar to those of the example embodiment 1, a plurality of second skin reflection regions SB1 to SB4 formed on the sheet member ST. Each of the plurality of first skin reflection regions SB1 to SB4 indicates a reflectance of a skin with respect to the second wavelength light.

Specifically, the test chart TC2 includes, in addition to the sheet member ST and a first region SA similar to those of the example embodiment 1, a second region SB being appropriately set on one principal plane of the sheet member ST. Specifically, the second region SB is set on a principal plane common to the one principal plane on which the first region SA is set.

The plurality of second skin reflection regions SB1 to SB4 are formed in the second region SB. Each of the plurality of second skin reflection regions SB1 to SB4 indicates a reflectance (unit is, for example, %) of a skin with respect to the second wavelength light. Each of the second skin reflection regions SB1 to SB4 is also referred to as a color chart, a patch, or the like. Note that, a plurality of (specifically, two or more) second skin reflection regions may be formed in the second region SB, and the number of the second skin reflection regions is not limited to four.

The second wavelength light is light for use in photographing of a subject. A wavelength of the second wavelength light is a second wavelength. For example, the second wavelength light is near infrared light, and the second wavelength in this case is, as well as first wavelength, a specific wavelength to be selected from among 700 to 2500 nm. However, the second wavelength is different from the first wavelength.

In an example illustrated in FIG. 13, the second skin reflection region SB1 is a region associated with a skin of a person whose reflectance with respect to the second wavelength light is B1. The second skin reflection region SB2 is a region associated with a skin of a person whose reflectance with respect to the second wavelength light is B2. The second skin reflection region SB3 is a region associated with a skin of a person whose reflectance with respect to the second wavelength light is B3. The second skin reflection region SB4 is a region associated with a skin of a person whose reflectance with respect to the second wavelength light is B4.

The reflectances B1 to B4 differ from one another. A magnitude of B1 to B4 according to the present example embodiment is B1>B2>B3>B4.

B1 is a maximum reflectance of a skin with respect to the second wavelength light. Specifically, the second skin reflection region SB1 is equivalent to a “second maximum region” indicating a maximum reflectance of a skin with respect to the second wavelength light.

B4 is a minimum reflectance of a skin with respect to the second wavelength light. Specifically, the second skin reflection region SB4 is equivalent to a “second minimum region” indicating a minimum reflectance of a skin with respect to the second wavelength light.

Note that, the plurality of second skin reflection regions SB1 to SB4 may include the second maximum region and the second minimum region, and the number of the second skin reflection regions may be changed appropriately, as far as the number is two or more.

There are various manufacturing methods of the test chart TC2, and, for example, it is possible to manufacture similarly to the test chart TC1 according to the example embodiment 1. Specifically, in a case where the test chart TC2 is manufactured, a worker may prepare, in addition to a plurality of pieces of paper on which each of the plurality of first skin reflection regions SA1 to SA4 is formed, a plurality of pieces of paper on which each of the plurality of second skin reflection regions SB1 to SB4 is formed, and paste the each piece of paper to a mount. Then, the worker may prepare black cardboard paper of the same size as a size of the mount, cut out a portion of the cardboard paper associated with the plurality of pieces of paper (specifically; the plurality of first skin reflection regions SA1 to SA4, and the plurality of second skin reflection regions SB1 to SB4) pasted to the mount, and paste together the black cardboard paper, and the mount to which the plurality of pieces of paper are pasted.

### (Configuration Example of Image Analysis System 200)

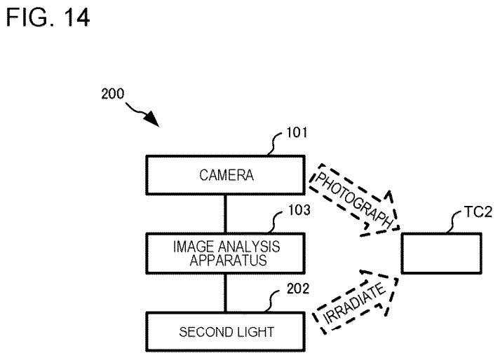

FIG. 14 is a diagram illustrating a configuration example of an image analysis system 200 according to the present example embodiment. The image analysis system 200 includes a second light 202, in place of the first light 102 according to the example embodiment 1. The image analysis system 200 may be configured similarly to the image analysis system 100 according to the example embodiment 1 except for this point.

The second light 202 is a light for irradiating the second wavelength light to a subject. In a case where an image acquired by photographing is used in face authentication, the second light 202 is provided, for example, in such a way as to irradiate the second wavelength light to a face of a person during a time when a camera 101 performs photographing. The second light 202 may irradiate the second wavelength light to the test chart TC2 in advance preparation before the camera 101 photographs a subject.

Note that, the image analysis system 200 may include a plurality of second lights 202.

The image analysis apparatus 103 according to the present example embodiment analyzes a chart image to be generated by photographing the test chart TC2, in place of the test chart TC1 in the example embodiment 1. Further, the image analysis apparatus 103 according to the present example embodiment uses a plurality of second skin reflection images SBim1 to SBim4, in place of the plurality of first skin reflection images SAim1 to SAim4 in the example embodiment 1. The second skin reflection images SBim1 to SBim4 are image portions each indicating the plurality of second skin reflection regions SB1 to SB4 in a chart image.

The image analysis apparatus 103 according to the present example embodiment may be functionally configured similarly to the example embodiment 1 except for these. Note that, regarding which one of the first skin reflection images SAim1 to SAim4, and the second skin reflection images SBim1 to SBim4 is used, for example, the image analysis apparatus 103 may automatically decide according to a type of the lights 102 and 202, and interchange the first skin reflection images SAim1 to SAim4, and the second skin reflection images SBim1 to SBim4, or may interchange the first skin reflection images SAim1 to SAim4, and the second skin reflection images SBim1 to SBim4 in response to a user instruction.

The image analysis apparatus 103 according to the present example embodiment may be physically configured similarly to the example embodiment 1.

In an image analysis method according to the present example embodiment, a chart image to be generated by photographing the test chart TC2 is analyzed, in place of the test chart TC1 in the example embodiment 1. Further, in the image analysis method according to the present example embodiment, the plurality of second skin reflection images SBim1 to SBim4 may be used, in place of the plurality of first skin reflection images SAim1 to SAim4 in the example embodiment 1.

The image analysis method according to the present example embodiment may be similar to that of the example embodiment 1 except for these.

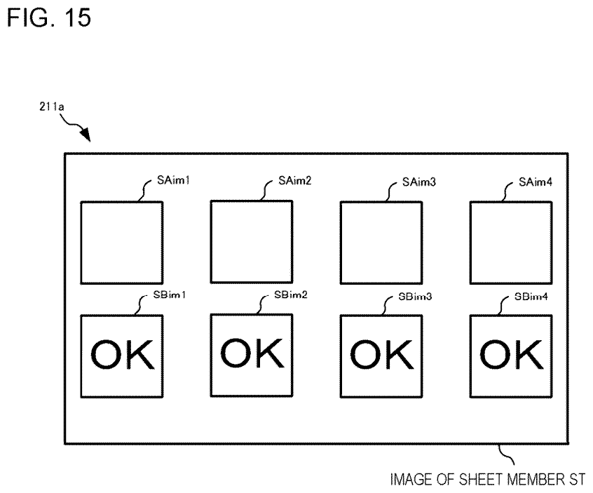

FIGS. 15 to 18 are diagrams illustrating one example of a display image 211 according to the present example embodiment. Each of FIGS. 15 to 18 is an example associated with FIGS. 9 to 12 that are referred to in the example embodiment 1.

Specifically, FIG. 15 illustrates one example of a display image 211a in a case where all the second skin reflection images SBim1 to SBim4 are decided to be a good image. As described in the example embodiment 1, by referring to the display image 211a, it is clear that intensity of the second wavelength light is appropriate in a light environment in a photographing place.

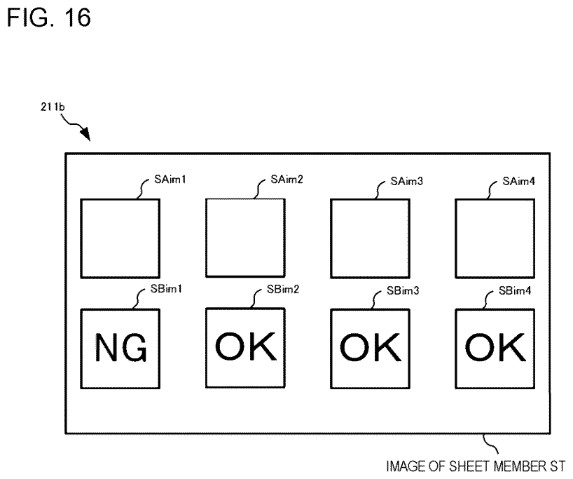

FIG. 16 illustrates one example of a display image 211b in a case where only the second skin reflection image SBim1 is decided not to be a good image. As described in the example embodiment 1, by referring to the display image 211b, it is clear that the second wavelength light may be too strong in a light environment in a photographing place.

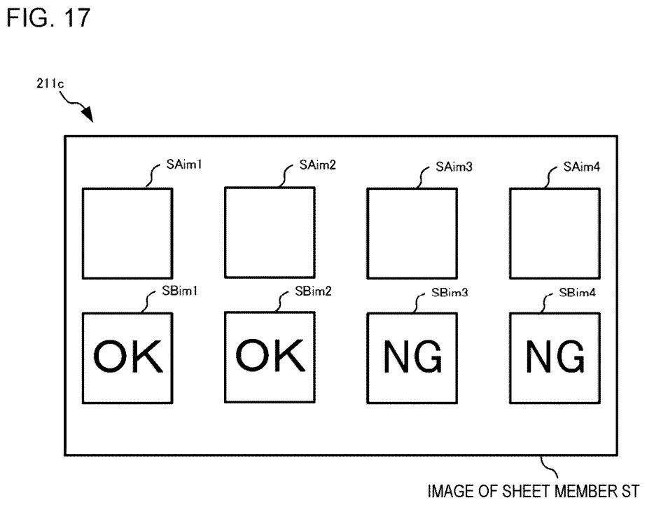

FIG. 17 illustrates one example of a display image 211c in a case where only the second skin reflection image SBim3 and the second skin reflection image SBim4 are decided not to be a good image. As described in the example embodiment 1, by referring to the display image 211c, it is clear that the second wavelength light may be weak in a light environment in a photographing place.

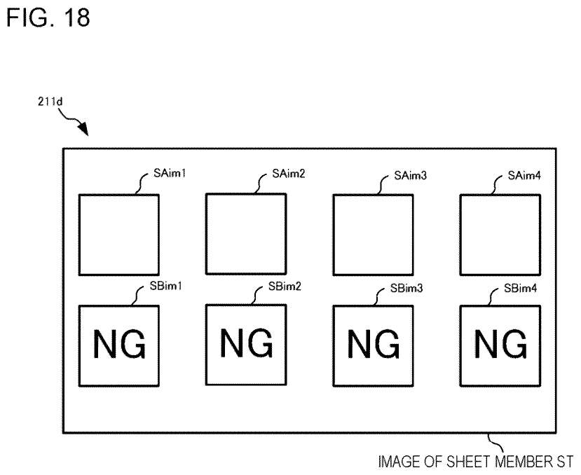

FIG. 18 illustrates one example of a display image 211d in a case where all the second skin reflection images SBim1 to SBim4 are decided not to be a good image. As described in the example embodiment 1, by referring to the display image 211d, it is clear that the second wavelength light may be very weak in a light environment in a photographing place.

According to the present example embodiment, the test chart TC2 further includes the plurality of second skin reflection regions SB1 to SB4 formed on the sheet member ST. Each of the second skin reflection regions SB1 to SB4 indicates a reflectance of a skin with respect to the second wavelength light.

This enables to check in advance whether to be able to acquire an appropriate image depending on an application, in a case where a subject is photographed, also in the image analysis systems 100 and 200 in which the first wavelength light and the second wavelength light are used, and handle in such a way that a light environment becomes appropriate, in a case where the light environment is inappropriate. Therefore, it becomes possible to acquire an appropriate skin image by photographing using light of a plurality of specific wavelengths.

According to the present example embodiment, the plurality of first skin reflection regions SA1 to SA4 include a first maximum region (first skin reflection region SA1) indicating a maximum reflectance of a skin with respect to the first wavelength light, and a first minimum region (first skin reflection region SA4) indicating a minimum reflectance of a skin with respect to the first wavelength light.

This enables to check in advance whether to be able to acquire an appropriate image depending on an application, irrespective of a reflectance of a skin of a subject, in a case where the subject is photographed by using the first wavelength light, and handle in such a way that a light environment becomes appropriate, in a case where the light environment is inappropriate. Therefore, it becomes possible to acquire an appropriate skin image by photographing using light of a specific wavelength.

The plurality of second skin reflection regions SB1 to SB4 include a second maximum region (second skin reflection region SB1) indicating a maximum reflectance of a skin with respect to the second wavelength light, and a second minimum region (second skin reflection region SB4) indicating a minimum reflectance of a skin with respect to the second wavelength light.

This enables to check in advance whether to be able to acquire an appropriate image depending on an application, irrespective of a reflectance of a skin of a subject, in a case where a subject is photographed by using the second wavelength light, and handle in such a way that a light environment becomes appropriate, in a case where the light environment is inappropriate. Therefore, it becomes possible to acquire an appropriate skin image by photographing using light of a plurality of specific wavelengths.

According to the present example embodiment, the first wavelength light is near infrared light.

This enables to check in advance whether to be able to acquire an appropriate image depending on an application, in a case where a subject is photographed by using near infrared light, and handle in such a way that a light environment becomes appropriate, in a case where the light environment is inappropriate. Therefore, it becomes possible to acquire an appropriate skin image by photographing using near infrared light.

According to the present example embodiment, the second wavelength light is also near infrared light.

This enables to check in advance whether to be able to acquire an appropriate image depending on an application, in a case where a subject is photographed by using near infrared light with any wavelength of a plurality of wavelengths, and handle in such a way that a light environment becomes appropriate, in a case where the light environment is inappropriate. Therefore, it becomes possible to acquire an appropriate skin image by photographing using near infrared light with any wavelength of a plurality of wavelengths.

### Example Embodiment 3

In the present example embodiment, a first example in which an image analysis apparatus adjusts a predetermined target device, based on a result acquired by analyzing a chart image is described. In the present example embodiment, a case where an image analysis apparatus as described above is applied in place of the image analysis apparatus 103 included in the image analysis system 100 according to the example embodiment 1 is described as an example. Therefore, in the present example embodiment, description overlapping the example embodiment 1 is omitted as necessary, and a point different from the example embodiment 1 is mainly described.

Note that, an image analysis apparatus 303 described in the present example embodiment may be applied in place of the image analysis apparatus 103 included in the image analysis system 200 according to the example embodiment 2.

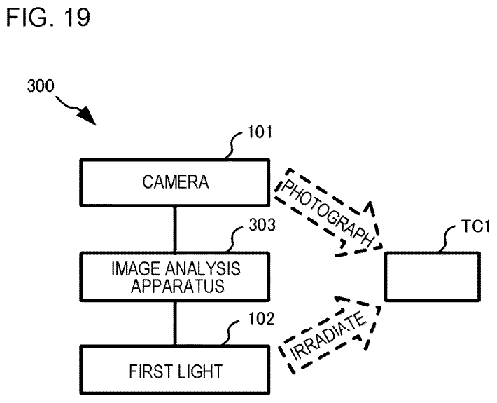

FIG. 19 is a diagram illustrating a configuration example of an image analysis system 300 according to an example embodiment 3. The image analysis system 300 includes the image analysis apparatus 303, in place of the image analysis apparatus 103. The image analysis system 300 may be configured similarly to the image analysis system 100 according to the example embodiment 1 except for this point.

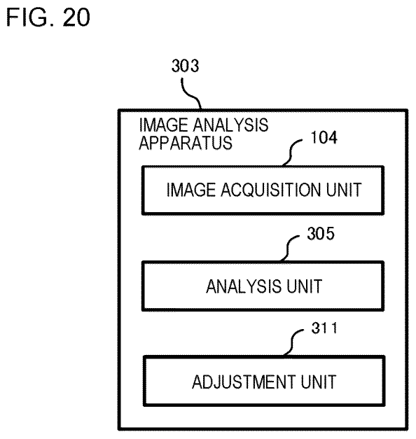

FIG. 20 is a diagram illustrating a functional configuration example of the image analysis apparatus 303 according to the example embodiment 3. The image analysis apparatus 303 includes an image acquisition unit 104 similar to that of the example embodiment 1, an analysis unit 305 in place of the analysis unit 105 according to the example embodiment 1, and an adjustment unit 311.

The analysis unit 305 analyzes a chart image similarly to the analysis unit 105 according to the example embodiment 1. The analysis unit 305 according to the present example embodiment derives brightness of each of a plurality of first skin reflection images SAim1 to SAim4.

The adjustment unit 311 adjusts a predetermined target device, based on a result acquired by analyzing a chart image.

Herein, a target device includes, for example, at least one of a device for use in photographing, and a device that affects a light environment in a photographing place. A device for use in photographing includes, for example, at least one of a camera 101, a first light 102 being an illumination for photographing, and the like. A device that affects a light environment in a photographing place includes, in a case where the photographing place is, for example, indoor, at least one of an illumination device such as an interior light that illuminates the photographing place, a device that operates a daylighting adjustment tool such as a curtain or a blind that adjusts daylighting from a window; and the like.

In a case where a target device includes the camera 101, the adjustment unit 311 may adjust a photographing condition (an aperture, a shutter speed, and the like) of the camera 101. In a case where a target device includes the first light 102, the adjustment unit 311 may adjust, for example, turning on and off of the first light 102, brightness in a case where the first light 102 is turned on, and the like. In a case where the first light 102 is plural, the adjustment unit 311 may adjust turning on and off of each of the first lights 102, brightness in a case where each of the first lights 102 is turned on, and the like.

In a case where a target device includes one or a plurality of illumination devices, the adjustment unit 311 may adjust turning on and off of each of the illumination devices, brightness in a case where each of the illumination devices is turned on, and the like. In a case where a target device includes a device that operates a daylighting adjustment tool, the adjustment unit 311 may adjust a daylight quantity from a window by operating the daylighting adjustment tool.

The adjustment unit 311 according to the present example embodiment adjusts a target device in such a way that brightness of the plurality of first skin reflection images SAim1 to SAim4 becomes equal to or less than brightness of a first maximum image, and becomes equal to or more than brightness of a first minimum image.

A first maximum image is an image indicating a first maximum region (first skin reflection region SA1) in a chart image, and is the first skin reflection image SAim1 in the present example embodiment. A first minimum image is an image indicating a first minimum region (first skin reflection region SA4) in a chart image, and is the first skin reflection image SAim4 in the present example embodiment.

The image analysis apparatus 303 according to the present example embodiment may be physically configured similarly to the image analysis apparatus 103 according to the example embodiment 1.

### (Example of Image Analysis Method According to Example Embodiment 3)

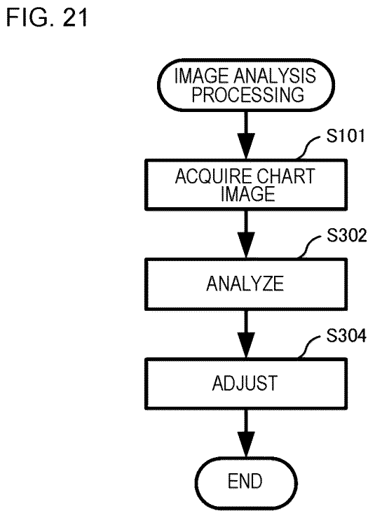

FIG. 21 is a flowchart illustrating one example of an image analysis method according to the example embodiment 3.

In a case where step S101 similar to that in the example embodiment 1 is executed, the analysis unit 305 analyzes a chart image (step S302).

In the present example embodiment, in step S302, the analysis unit 305 derives brightness of each of the plurality of first skin reflection images SAim1 to SAim4.

The adjustment unit 311 adjusts a predetermined target device, based on a result acquired by analyzing the chart image in step S302 (step S304), and finishes image analysis processing.

Specifically, for example, in step S304, the adjustment unit 311 adjusts a target device in such a way that brightness of the plurality of first skin reflection images SAim1 to SAim4 becomes equal to or less than brightness of the first maximum image, and becomes equal to or more than brightness of the first minimum image.

As described in the example embodiment 1, reflectances A1 to A4 of the first skin reflection regions SA1 to SA4 have a relationship: A1>A2>A3>A4. Therefore, in a case where a light environment in a photographing place is appropriate, brightness of the plurality of first skin reflection images SAim1 to SAim4 becomes brightness of the first skin reflection image SAim1>brightness of the first skin reflection image SAim2>brightness of the first skin reflection image SAim3>brightness of the first skin reflection image SAim4. Specifically, at least, brightness of the plurality of first skin reflection images SAim1 to SAim4 becomes equal to or less than brightness of the first maximum image, and becomes equal to or more than brightness of the first minimum image.

In the image analysis processing according to the present example embodiment, a target device is adjusted in such a way that brightness of the plurality of first skin reflection images SAim1 to SAim4 becomes equal to or less than brightness of the first maximum image (first skin reflection image SAim1), and becomes equal to or more than brightness of the first minimum image (first skin reflection image SAim4). This enables to adjust in such a way that a light environment in a photographing place becomes appropriate. Therefore, it becomes possible to acquire an appropriate skin image by photographing using light of a specific wavelength.

### (Advantageous Effect)

According to the present example embodiment, the image analysis apparatus 303 further includes the adjustment unit 311 that adjusts a predetermined target device, based on an analysis result of a chart image. The analysis unit 305 derives brightness of the plurality of first skin reflection images SAim1 to SAim4 each indicating the plurality of first skin reflection regions SA1 to SA4. The adjustment unit 311 adjusts a target device in such a way that brightness of the plurality of first skin reflection images SAim1 to SAim4 becomes equal to or less than brightness of the first maximum image (first skin reflection image SAim1) indicating a first maximum region, and becomes equal to or more than brightness of the first minimum image (first skin reflection image SAim4) indicating a first minimum region.

This enables to adjust in such a way that a light environment in a photographing place becomes appropriate. Therefore, it becomes possible to acquire an appropriate skin image by photographing using light of a specific wavelength.

### Example Embodiment 4

In the present example embodiment, a second example in which an image analysis apparatus adjusts a predetermined target device, based on a result acquired by analyzing a chart image is described. In the present example embodiment, a case where an image analysis apparatus as described above is applied in place of the image analysis apparatus 103 included in the image analysis system 100 according to the example embodiment 1 is described as an example. Therefore, in the present example embodiment, description overlapping the example embodiment 1 is omitted as necessary, and a point different from the example embodiment 1 is mainly described.

Note that, an image analysis apparatus 403 described in the present example embodiment may be applied in place of the image analysis apparatus 103 included in the image analysis system 200 according to the example embodiment 2.

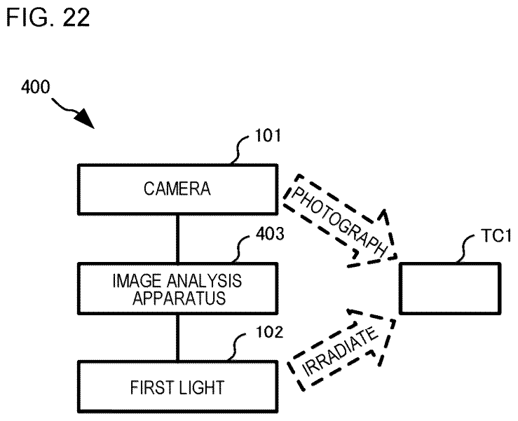

FIG. 22 is a diagram illustrating a configuration example of an image analysis system 400 according to an example embodiment 4. The image analysis system 400 includes the image analysis apparatus 403, in place of the image analysis apparatus 103. The image analysis system 400 may be configured similarly to the image analysis system 100 according to the example embodiment 1 except for this point.

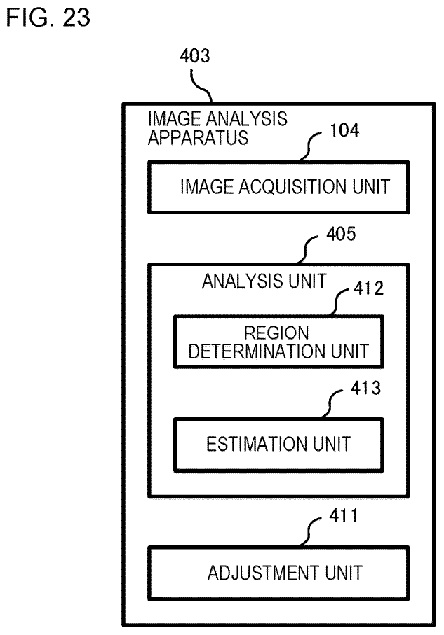

FIG. 23 is a diagram illustrating a functional configuration example of the image analysis apparatus 403 according to the example embodiment 4. The image analysis apparatus 403 includes an image acquisition unit 104 similar to that of the example embodiment 1, an analysis unit 405 in place of the analysis unit 105 according to the example embodiment 1, and an adjustment unit 411.

The analysis unit 405 analyzes a chart image similarly to the analysis unit 105 according to the example embodiment 1. The analysis unit 405 according to the present example embodiment divides a chart image into a plurality of regions, and estimates a light environment in a photographing place of a test chart, based on each region.

Specifically, for example, the analysis unit 405 includes a region determination unit 412, and an estimation unit 413.

The region determination unit 412 determines a first image region and a second image region in a chart image, based on a predetermined region determination condition.

A region determination condition is a condition for determining, from a chart image, a plurality of image regions to be used for estimating a light environment in a photographing place. The region determination condition is defined in association with at least one of an illumination provided for photographing, a position of each of one or a plurality of illumination devices provided in a photographing place, and ambient light in the photographing place.

An illumination provided for photographing is, for example, a first light 102 or the like according to the present example embodiment. One or a plurality of illumination devices provided in a photographing place are, for example, an interior light or the like that illuminates the photographing place, in a case where the photographing place is indoor. Ambient light in a photographing place is, for example, sunlight entering a room from a window; in a case where the photographing place is indoor. Since sunlight entering a room from a window may change depending on a time period, the region determination condition may include a time period, or may also include, for example, a condition for determining a plurality of image regions being different for each time period.

The estimation unit 413 estimates a light environment in a photographing place of a test chart TC1, based on each of the first image region and the second image region.

Specifically, for example, the estimation unit 413 estimates whether each of a first light environment and a second light environment is good in light of a predetermined second decision criterion. A first light environment is a light environment associated with the first image region. A second light environment is a light environment associated with the second image region.

A second decision criterion is a criterion for deciding whether a light environment is good. The second decision criterion includes, for example, a range of brightness of an image associated with a good light environment. In a case where brightness of an image region lies within a range defined by the second decision criterion, the estimation unit 413 may estimate that a light environment associated with the image region is good. In a case where brightness of an image region is out of the range defined by the second decision criterion, the estimation unit 413 may estimate that a light environment associated with the image region is not good.

For example, in a case where it is decided whether a light environment is an environment that may cause black crushing or overexposure in a chart image, the second decision criterion may be defined by using a first threshold value and a second threshold value similarly to a first decision criterion. In this case, the second decision criterion may be such that brightness of an image region is less than the first threshold value, and more than the second threshold value.

Similarly to the adjustment unit 311 according to the example embodiment 3, the adjustment unit 411 adjusts a predetermined target device, based on a result acquired by analyzing a chart image. The adjustment unit 411 according to the present example embodiment adjusts a target device, based on a light environment estimated by using the estimation unit 413.

The image analysis apparatus 403 according to the present example embodiment may be physically configured similarly to the image analysis apparatus 103 according to the example embodiment 1.

### (Example of Image Analysis Method According to Example Embodiment 4)

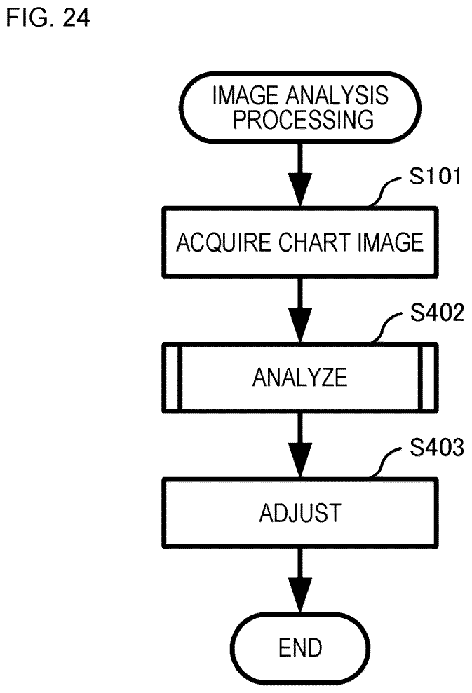

FIG. 24 is a flowchart illustrating one example of an image analysis method according to the example embodiment 4.

In a case where step S101 similar to that in the example embodiment 1 is executed, the analysis unit 405 analyzes a chart image (step S402).

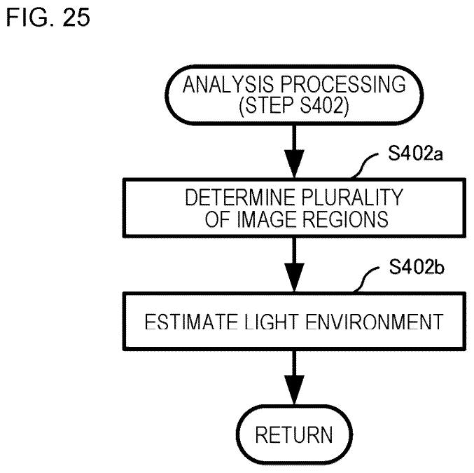

FIG. 25 is a flowchart illustrating a detailed example of analysis processing (step S402) according to the example embodiment 4.

The region determination unit 412 determines the first image region and the second image region in a chart image, based on a predetermined region determination condition (step S402a).

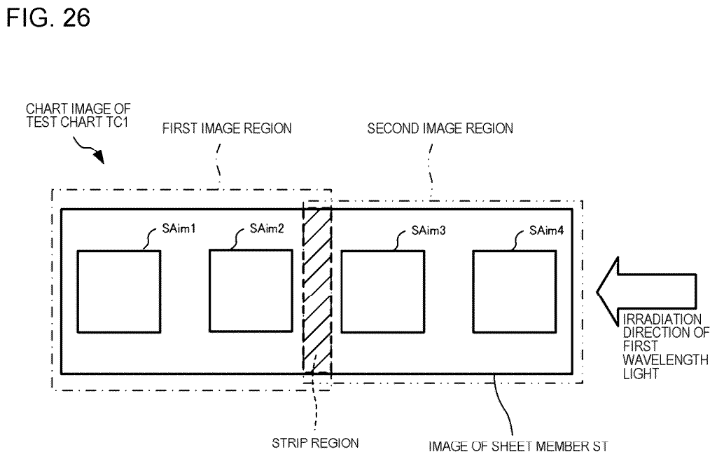

FIG. 26 is a diagram illustrating a first example of the first image region and the second image region in a case where a region determination condition associated with a position where the first light 102 is installed is set. FIG. 26 is one example of a the first image region and the second image region in a case where the first light 102 irradiates first wavelength light from a right side of the test chart TC1.

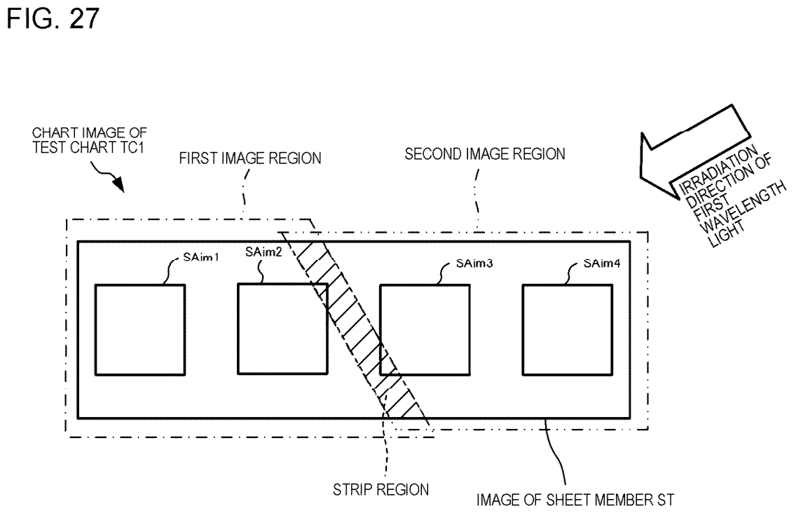

FIG. 27 is a diagram illustrating a second example of the first image region and the second image region in a case where a region determination condition associated with a position where the first light 102 is installed is set. FIG. 27 is one example of the first image region and the second image region in a case where the first light 102 irradiates the first wavelength light from an upper right side of the test chart TC1.

In the examples in FIGS. 26 and 27, a region determination condition includes, for example, setting a strip region of a predetermined width in which a strip region of a predetermined width passes through a substantially center of the chart image substantially vertical with respect to a direction in which the first light 102 irradiates the first wavelength light. Further, the region determination condition includes, for example, determining the first image region and the second image region on a side opposite to each other via the strip region.

Note that, in the examples in FIGS. 26 and 27, an example in which a part of the first image region and the second image region is different from each other is described, however, the entirety of the first image region and the second image region may be different from each other.

FIG. 25 is referred to again.

The estimation unit 413 estimates a light environment in a photographing place of the test chart TC1, based on each of the first image region and the second image region determined in step S402a (step S402b), and returns to image analysis processing.

Specifically, for example, the estimation unit 413 derives brightness of each of the first image region and the second image region determined in step S402a. The estimation unit 413 estimates a light environment in a photographing place of the test chart TC1, based on brightness of each of the first image region and the second image region, and the second decision criterion.

It is assumed that the second decision criterion is that brightness of an image region is less than the first threshold value, and more than the second threshold value. In this case, the estimation unit 413 decides, regarding each of the first image region and the second image region, whether brightness is less than the first threshold value, and more than the second threshold value.

For example, in a case where brightness of the first image region is less than the first threshold value, and more than the second threshold value, in the estimation unit 413, a possibility of occurrence of overexposure, black crushing, or the like in the first image region is low. Therefore, the estimation unit 413 estimates that the first light environment associated with the first image region is good.

Further, for example, in a case where brightness of the first image region is equal to or more than the first threshold value, or in a case where brightness of the first image region is equal to or less than the second threshold value, in the estimation unit 413, a possibility of occurrence of overexposure, black crushing, or the like in the first image region is high. Therefore, the estimation unit 413 estimates that the first light environment associated with the first image region is not good.

Similarly to the above-described first light environment, the estimation unit 413 also estimates whether the second light environment associated with the second image region is good, based on brightness of the second image region, and the second decision criterion.

FIG. 24 is referred to again.

The adjustment unit 411 adjusts a predetermined target device, based on a result acquired by analyzing a chart image in step S402 (step S403).

The adjustment unit 411 according to the present example embodiment adjusts a target device, based on a light environment estimated in step S402. Various pieces of processing may be available as the adjustment processing (step S403), and examples thereof are described in the following.

### (Example 1 of Adjustment Processing According to Example Embodiment 4)

Specifically, for example, it is assumed that one light environment between the first light environment and the second light environment is estimated to be good, and the other light environment between the first light environment and the second light environment is estimated not to be good. Specifically, for example, it is assumed that the first light environment is estimated not to be good, and the second light environment is estimated to be good, based on each of the first image region and the second image region illustrated in FIG. 26.

In this case, the adjustment unit 411 may apply an operation condition of a target device associated with the second light environment being the one light environment to an operation condition of the target device associated with the first light environment being the other light environment.

Herein, a target device may be set in advance in association with the first light environment and the second light environment. For example, in a case where a target device is an illumination device such as an interior light, an illumination device located on a left side of the test chart TC1 may be associated with the first light environment, and an illumination device located on a right side of the test chart TC1 may be associated with the second light environment.

This enables to make the first light environment close to the second light environment, and make the first light environment a good environment.

### (Example 2 of Adjustment Processing According to Example Embodiment 4)

Specifically, for example, it is assumed that one light environment between the first light environment and the second light environment is estimated to be good, and the other light environment between the first light environment and the second light environment is estimated not to be good. Specifically, for example, it is assumed that the first light environment is estimated not to be good, and the second light environment is estimated to be good, based on each of the first image region and the second image region illustrated in FIG. 26.

In this case, the adjustment unit 411 may adjust a target device in such a way as to make the first light environment being the other light environment close to the second light environment being the one light environment. Similarly to the above-described example 1, a target device may be set in advance in association with the first light environment and the second light environment. For example, the adjustment unit 411 may gradually adjust a target device associated with one or both of the first light environment and the second light environment in such a way as to make the first light environment being the other light environment close to the second light environment being the one light environment.

This enables to make the first light environment close to the second light environment, and make the first light environment a good environment.

Note that, in both of the adjustment processing examples 1 and 2, a part or all of target devices may be a plurality of first lights 102, for example, in a case where the plurality of first lights 102 that irradiate the test chart TC1 from a different direction are provided.

### (Advantageous Effect)

According to the present example embodiment, the image analysis apparatus 403 further includes the adjustment unit 411 that adjusts a predetermined target device, based on an analysis result of a chart image. The analysis unit 405 includes the region determination unit 412, and the estimation unit 413. The region determination unit 412 determines the first image region and the second image region in the chart image, based on a predetermined region determination condition. The estimation unit 413 estimates a light environment in a photographing place of the test chart TC1, based on each of the first image region and the second image region. The adjustment unit 411 adjusts the target device, based on the estimated light environment.

This enables to adjust a target device in such a way as to make a light environment associated with each of the first image region and the second image region good. Therefore, it becomes possible to acquire an appropriate skin image by photographing using light of a specific wavelength.

According to the present example embodiment, a region determination condition is defined in association with at least one of an illumination provided for photographing, a position of each of one or a plurality of illumination devices provided in a photographing place, and ambient light in the photographing place.

This enables to determine the first image region and the second image region according to a photographing environment, and handle in such a way that a light environment associated with each image region is evaluated and a light environment becomes appropriate, in a case where the light environment is inappropriate. Therefore, it becomes possible to efficiently acquire an appropriate skin image by photographing using light of a specific wavelength.

According to the present example embodiment, the estimation unit 413 estimates whether each of the first environment associated with the first image region, and the second light environment associated with the second image region is good in light of a predetermined decision criterion (second decision criterion). In a case where it is estimated that one light environment between the first light environment and the second light environment is good, and it is estimated that the other light environment is not good, the adjustment unit 411 applies an operation condition of a target device associated with the one light environment to an operation condition of the target device associated with the other light environment.

This enables to make the first light environment close to the second light environment, and make the first light environment a good environment. For example, in a case where a plurality of illumination devices as a target device are present, it is possible to achieve reduction of time and effort for adjusting each of the illumination devices. Therefore, it becomes possible to efficiently acquire an appropriate skin image by photographing using light of a specific wavelength.

According to the present example embodiment, the estimation unit 413 estimates whether each of the first light environment associated with the first image region, and the second light environment associated with the second image region is good in light of a predetermined decision criterion (second decision criterion). In a case where it is estimated that one light environment between the first light environment and the second light environment is good, and it is estimated that the other light environment is not good, the adjustment unit 411 adjusts a target device in such a way as to make the other light environment close to the one light environment.

This enables to make the first light environment close to the second light environment, and make the first light environment a good environment. For example, in a case where a plurality of illumination devices as a target device are present, it is possible to achieve reduction of time and effort for adjusting each of the illumination devices. Therefore, it becomes possible to efficiently acquire an appropriate skin image by photographing using light of a specific wavelength.

### Example Embodiment 5

In the present example embodiment, an example in which a test chart includes a plurality of gray regions for evaluating a dynamic range in a test image is described. In the present example embodiment, an example in which a plurality of gray regions include a plurality of first gray regions and a plurality of second gray regions is described.

In the present example embodiment, a case where a test chart as described above is applied in place of the test chart TC1 included in the image analysis system 100 according to the example embodiment 1 is described as an example. Therefore, in the present example embodiment, description overlapping the example embodiment 1 is omitted as necessary, and a point different from the example embodiment 1 is mainly described.

Note that, a test chart TC3 described in the present example embodiment may be applied in place of the test charts TC1 and TC2 included in the image analysis systems 200, 300, and 400 according to the example embodiments other than the example embodiment 1. Further, a plurality of gray regions may include only either one of first gray regions GLA1 to GLA10, and second gray regions GLB1 to GLB2.

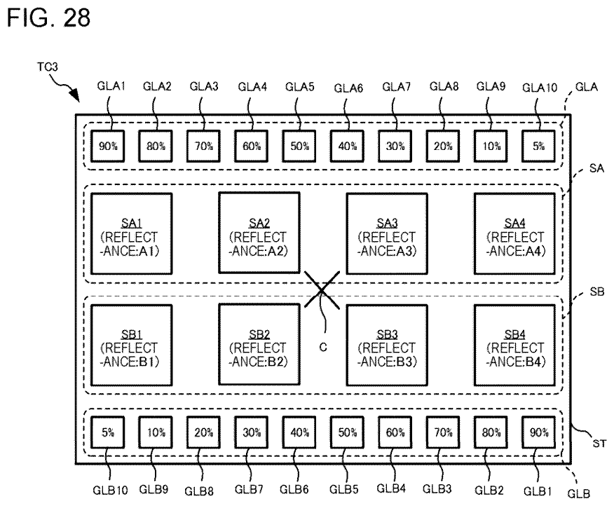

FIG. 28 is a diagram illustrating a configuration example of the test chart TC3 according to an example embodiment 5. The test chart TC3 includes a sheet member ST and a plurality of first skin reflection regions SA1 to SA4 similar to those of the example embodiment 1, and a plurality of second skin reflection regions SB1 to SB4 similar to those of the example embodiment 2. The test chart TC3 further includes the plurality of first gray regions GLA1 to GLA10, and the plurality of second gray regions GLB1 to GLB10.

Specifically, the test chart TC3 includes, in addition to the sheet member ST and a first region SA similar to those of the example embodiment 1, and a second region SB similar to that of the example embodiment 2, a third region GLA and a fourth region GLB that are appropriately set on one principal plane of the sheet member ST. Specifically, the third region GLA and the fourth region GLB are set on a principal plane common to one principal plane on which the first region SA and the second region SB are set.

The plurality of first gray regions GLA1 to GLA10 are formed in the third region GLA. Each of the plurality of first gray regions GLA1 to GLA10 indicates a reflectance (unit is, for example, %) of a skin with respect to first wavelength light and second wavelength light. Each of the first gray regions GLA1 to GLA10 is also referred to as a color chart, a patch, or the like.

The first gray regions GLA1 to GLA10 indicate depth different from one another in white and black. The first gray regions GLA1 to GLA10 are arranged in order of depth in such a way that depth indicated by each thereof changes constantly.

In an example illustrated in FIG. 28, the first gray regions GLA1 to GLA10 are gray regions in each of which a reflectance is 90%, 80%, 70%, 60%, 50%, 40%, 30%, 20%, 10%, and 5%. The first gray regions GLA1 to GLA10 are arranged at equal intervals in a horizontal direction in such a way that depth gradually increases (a reflectance decreases) from left to right in FIG. 28.

Note that, a difference in depth between GLA9 and GLA10 is different from a difference (10%) in depth of the first gray regions GLA1 to GLA9 other than the above and adjacent to each other, and is 5%, however, this is due to a manufacturing constraint, and is an example of the first gray region GLA10 whose reflectance is close to 0%.

The plurality of second gray regions GLB1 to GLB10 are formed in the fourth region GLB. Each of the plurality of second gray regions GLB1 to GLB10 indicates a reflectance (unit is, for example, %) of a skin with respect to the first wavelength light and the second wavelength light. Each of the second gray regions GLB1 to GLB10 is also referred to as a color chart, a patch, or the like.

The second gray regions GLB1 to GLB10 indicate depth associated with depth indicated by each of the plurality of first gray regions GLA1 to GLA10. Therefore, the plurality of first gray regions GLA1 to GLA10 and the plurality of second gray regions GLB1 to GLB10 include a plurality of combinations of a first gray region and a second gray region (e.g., GLA1 and GLB1, GLA2 and GLB2, GLA3 and GLB3, and the like) indicating the same depth.

The first gray regions GLA1 to GLA10 and the second gray regions GLB1 to GLB10 indicating associated depth are formed at a symmetric position with respect to a common point C. For example, GLA1 and GLB1, GLA2 and GLB2, GLA3 and GLB3, and the like are formed at a position with an equal distance in a direction opposite to each other with respect to the common point C. This allows the second gray regions GLB1 to GLB10 to be arranged in reverse order of depth with respect to the plurality of first gray regions GLA1 to GLA10 in such a way that depth indicated by each thereof changes constantly.

The point C is, for example, a center of the sheet member ST, but is not limited thereto.

In the example illustrated in FIG. 28, the second gray regions GLB1 to GLB10 are gray regions in each of which a reflectance is 90%, 80%, 70%, 60%, 50%, 40%, 30%, 20%, 10%, and 5%.

The first gray regions GLA1 to GLA10 are arranged at equal intervals in the horizontal direction in such a way that depth gradually increases (a reflectance decreases) from right to left in FIG. 28. In this way, the first gray regions GLA1 to GLA10 are arranged in the horizontal direction in reverse order of depth with respect to the plurality of first gray regions GLA1 to GLA10.

The plurality of first skin reflection regions SA1 to SA4, SB1 to SB4, and the plurality of second skin reflection regions SB1 to SB4 are formed between the plurality of first gray regions GLA1 to GLA10, and the plurality of second gray regions GLB1 to GLB10.

There are various manufacturing methods of the test chart TC3, and, for example, it is possible to manufacture similarly to the test charts TC1 and TC2 according to the example embodiment 1 or 2. Specifically, in a case where the test chart TC3 is manufactured, a worker prepares a plurality of pieces of paper on which each of the plurality of first skin reflection regions SA1 to SA4 and the plurality of second skin reflection regions SB1 to SB4 is formed. In addition to these, the worker prepares a plurality of pieces of paper on which each of the plurality of first gray regions GLA1 to GLA10 and the plurality of second gray regions GLB1 to GLB10 is formed. Then, the worker may prepare black cardboard paper of the same size as a size of a mount, and cut out a portion of the cardboard paper associated with the plurality of pieces of paper (specifically; the plurality of first skin reflection regions SA1 to SA4, the plurality of second skin reflection regions SB1 to SB4, the plurality of first gray regions GLA1 to GLA10, and the plurality of second gray regions GLB1 to GLB10) pasted to the mount. Then, the worker may paste together the black cardboard paper, and the mount to which the plurality of pieces of paper are pasted. Paint, for example, whose reflectance becomes substantially constant by near infrared light including the first wavelength light and the second wavelength light may be coated on the plurality of pieces of paper on which each of the first gray regions GLA1 to GLA10 and each of the second gray regions GLB1 to GLB10 is formed.

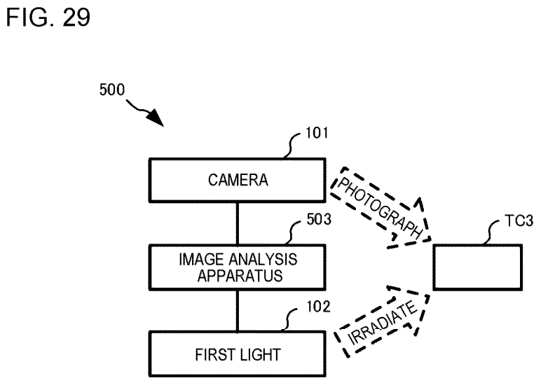

FIG. 29 is a diagram illustrating a configuration example of an image analysis system 500 according to the example embodiment 5. The image analysis system 500 includes an image analysis apparatus 503, in place of the image analysis apparatus 103 according to the example embodiment 1. The image analysis system 500 may be configured similarly to the image analysis system 100 according to the example embodiment 1 except for this point.

Note that, since the test chart TC3 includes the plurality of second skin reflection regions SB1 to SB4, the image analysis system 500 may include a second light 202 according to the example embodiment 2, in place of a first light 102.

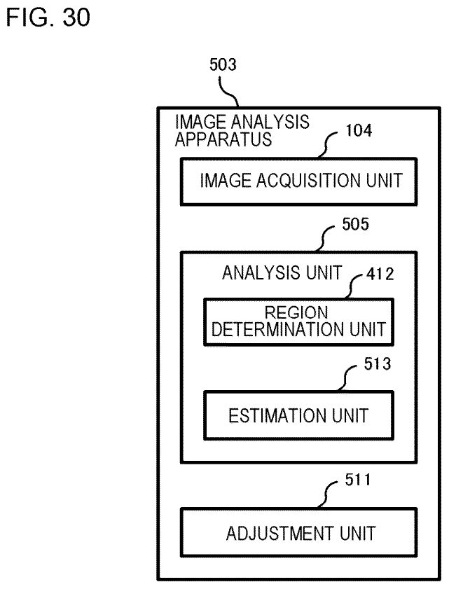

FIG. 30 is a diagram illustrating a functional configuration example of the image analysis apparatus 503 according to the example embodiment 5. The image analysis apparatus 503 functionally includes, for example, an image acquisition unit 104 similar to that of the example embodiment 1, an analysis unit 505, and an adjustment unit 511.

The analysis unit 505 analyzes a chart image similarly to the example embodiment 1. The analysis unit 105 functionally includes a region determination unit 412 similar to that of the example embodiment 4, and an estimation unit 513.

The estimation unit 513 estimates a light environment in a photographing place of the test chart TC3, based on each of a first image region and a second image region.

Specifically, for example, the estimation unit 413 estimates whether a light environment includes oblique light, based on brightness of a gray image group included in at least one of the first image region and the second image region. A gray image group is images indicating, in a chart image, each of the plurality of first gray regions and the plurality of second gray regions.

The estimation unit 413 estimates that a light environment includes oblique light, in a case where brightness of a gray image group is not brightness according to associated depth of the plurality of first gray regions GLA1 to GLA10, and the plurality of second gray regions GLB1 to GLB10.

Similarly to the adjustment unit 311 according to the example embodiment 4, the adjustment unit 511 adjusts a predetermined target device, based on a result acquired by analyzing a chart image. The adjustment unit 511 according to the present example embodiment adjusts a target device, based on a light environment estimated by using the estimation unit 513.

For example, in a case where the estimation unit 513 estimates that a light environment includes oblique light, there is a possibility that brightness of a chart image becomes uneven due to oblique light. Therefore, for example, the adjustment unit 511 adjusts a target device in such a way that brightness of a chart image becomes even.

The image analysis apparatus 503 according to the present example embodiment may be physically configured similarly to the image analysis apparatus 103 according to the example embodiment 1.

### (Example of Image Analysis Method According to Example Embodiment 5)

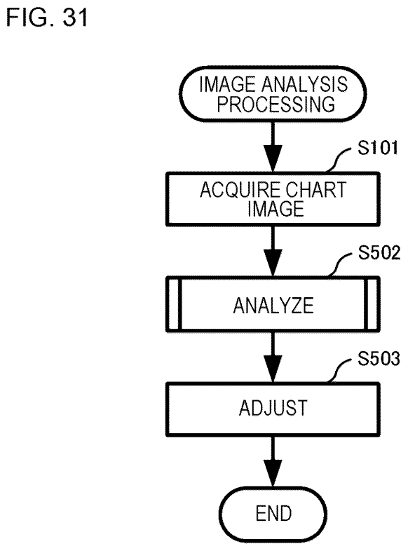

FIG. 31 is a flowchart illustrating one example of an image analysis method according to the example embodiment 5.

In a case where step S101 similar to that in the example embodiment 1 is executed, the analysis unit 505 analyzes a chart image (step S502).

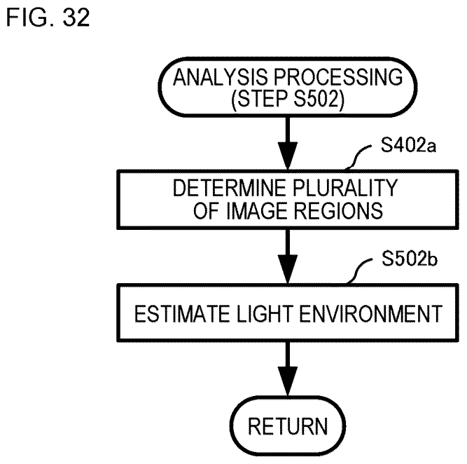

FIG. 32 is a flowchart illustrating a detailed example of analysis processing (step S502) according to the example embodiment 5.

The region determination unit 412 executes step S402a similar to that in the example embodiment 4.

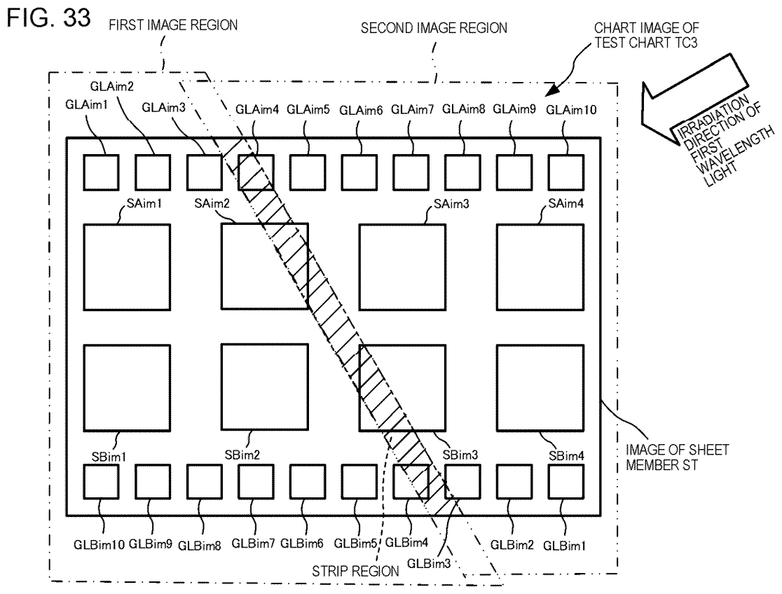

FIG. 33 is a diagram illustrating a third example of the first image region and the second image region in a case where a region determination condition associated with a position where the first light 102 is installed is set. FIG. 33 is one example of the first image region and the second image region in a case where the first light 102 irradiates the first wavelength light from an upper right side of the test chart TC3.

In FIG. 33, as described in the example embodiment 1, first skin reflection images SAim1 to SAim4 are image portions each indicating the plurality of first skin reflection regions SA1 to SA4 in a chart image of the test chart TC3. Second skin reflection images SBim1 to SBim4 are image portions each indicating the plurality of second skin reflection regions SB1 to SB4 in the chart image of the test chart TC3.

Further, in FIG. 33, first gray images GLAim1 to GLAim10 are image portions each indicating the plurality of first gray regions GLA1 to GLA10 in the chart image of the test chart TC3. Second gray images GLBim1 to GLBim10 are image portions each indicating the plurality of second gray regions GLB1 to GLB10 in the chart image of the test chart TC3. In the test chart TC3, the first gray regions GLA1 to GLA10 and the second gray regions GLB1 to GLB10 indicating associated depth are formed at a symmetric position with respect to the common point C. Therefore, as illustrated in FIG. 33, each of the first image region and the second image region includes an image in a darkest gray region and an image in a lightest gray region in the plurality of first gray regions GLA1 to GLA10 and the plurality of second gray regions GLB1 to GLB10.

An image in a darkest gray region is an image (first gray image GLAim10) in the first gray region GLA1, and an image (second gray image GLBim10) in the second gray region GLB1. An image in a lightest gray region is an image (first gray image GLAim1) in the first gray region GLA10, and an image (second gray image GLBim1) in the second gray region GLB10.

In the present example embodiment, the first image region includes one (second gray image GLBim10) of the images in the darkest gray region, and one (first gray image GLAim1) of the images in the lightest gray region. The second image region includes the other (first gray image GLAim10) of the images in the darkest gray region, and the other (second gray image GLBim1) of the images in the lightest gray region.

Further, in the present example embodiment, each of the first image region and the second image region includes each gray image of depth indicated by the plurality of first gray regions GLA1 to GLA10, and the plurality of second gray regions GLB1 to GLB10.

Specifically, the first image region includes, as images of gray regions in each of which a reflectance is 90%, 80%, 70%, 60%, 50%, 40%, 30%, 20%, 10%, and 5%, the first gray images GLAim1 to GLAim3, and the second gray images GLBim4 to GLBim10. The second image region includes, as images of gray regions in each of which a reflectance is 90%, 80%, 70%, 60%, 50%, 40%, 30%, 20%, 10%, and 5%, the second gray images GLBim1 to GLBim3, and the first gray images GLAim4 to GLAim10.

FIG. 32 is referred to again.

The estimation unit 513 estimates a light environment in a photographing place of the test chart TC3, based on each of the first image region and the second image region determined in step S402a (step S502b), and returns to image analysis processing.

In a case where brightness of a gray image group is not brightness according to associated depth of the plurality of first gray regions GLA1 to GLA10 and the plurality of second gray regions GLB1 to GLB10, the estimation unit 513 according to the present example embodiment estimates that a light environment includes oblique light.

For example, the estimation unit 513 estimates whether a light environment includes oblique light, based on an image in a darkest gray region and an image in a lightest gray region included in each of the first image region and the second image region. Specifically, for example, in a case where overexposure or black crushing has occurred in an image in a darkest gray region and an image in a lightest gray region included in only one of the first image region and the second image region, the estimation unit 513 estimates that a light environment includes oblique light.

Further, for example, the estimation unit 513 estimates whether a light environment includes oblique light, based on whether a difference in brightness of a gray image adjacent to each other is constant, regarding each of the first image region and the second image region.

In the present example embodiment, as described above, since a difference in reflectance of the first gray regions GLA1 to GLA10 adjacent to each other is constant, unless oblique light is present, a difference in brightness of the first gray images GLAim1 to GLAim10 adjacent to each other becomes substantially constant. Likewise, as described above, since a difference in reflectance of the second gray regions GLB1 to GLB10 adjacent to each other is constant, unless oblique light is present, a difference in brightness of the second gray images GLBim1 to GLBim10 adjacent to each other becomes substantially constant.

Therefore, for example, in a case where a difference in brightness of gray images adjacent to each other is not constant in at least one of the first image region and the second image region, the estimation unit 513 estimates that a light environment includes oblique light. For example, in a case where a difference in brightness of gray images adjacent to each other is constant in both of the first image region and the second image region, the estimation unit 513 estimates that a light environment does not include oblique light.

FIG. 31 is referred to again.

The adjustment unit 511 adjusts a predetermined target device, based on a result acquired by analyzing a chart image in step S502 (step S503).

Specifically, for example, in a case where it is estimated that a light environment includes oblique light in step S502, the adjustment unit 511 adjusts a target device in such a way that brightness of a chart image becomes even. For example, the adjustment unit 511 adjusts a target device associated with a bright image region in such a way that the bright image region due to oblique light is darkened between the first image region and the second image region. Further, for example, the adjustment unit 511 adjusts a target device associated with a dark image region in such a way that the dark image region due to oblique light is brightened between the first image region and the second image region.

Specifically, for example, it is assumed that it is estimated that a light environment does not include oblique light in step S502. In this case, the adjustment unit 511 adjusts a photographing condition of a camera 101 in such a way as to achieve an appropriate dynamic range that satisfies a predetermined condition, based on the first gray images GLA image 1 to GLA image 10, and the second gray images GLBim1 to GLBim10.

In the image analysis processing according to the present example embodiment, in a case where a light environment includes oblique light, it is possible to adjust a target device in such a way as to reduce an influence of oblique light on a chart image. This enables to acquire an appropriate skin image by photographing using light of a specific wavelength.

Further, in a case where a light environment does not include oblique light, it is possible to adjust a dynamic range. This also enables to acquire an appropriate skin image by photographing using light of a specific wavelength.

### (Advantageous Effect)

According to the present example embodiment, the test chart TC3 further includes the plurality of first gray regions GLA1 to GLA10 that are formed side by side on the sheet member ST, and indicate depth different from one another.

This enables to evaluate and adjust a gradation of the camera 101 by using the first gray images GLAim1 to GLAim10. Further, it is possible to evaluate or decide whether to be able to acquire a good image of a skin of a person to an extent that can be used in face authentication and the like by using the first skin reflection images SAim1 to SAim4 or the second skin reflection images SBim1 to SBim4. Specifically, it is possible, by using the one test chart TC3, to evaluate and adjust a gradation of the camera 101, and evaluate or decide whether to be able to acquire a good image of a skin of a person. Therefore, it becomes possible to acquire an appropriate skin image by photographing using light of a specific wavelength more easily than a case, for example, where an individual test chart is used for gradation of the camera 101 and for evaluation of an image of a skin of a person.

According to the present example embodiment, the test chart TC3 further includes the plurality of second gray regions GLB1 to GLB10 that are formed on the sheet member ST, and indicate depth associated with depth indicated by each of the plurality of first gray regions GLA1 to GLA10. The plurality of first gray regions GLA1 to GLA10 are arranged in order of depth in such a way that the depth indicated by each thereof changes with a certain difference. A first gray region and a second gray region indicating associated depth are formed at a symmetric position with respect to the common point C.

This makes it easy to include each gray image of depth indicated by the plurality of first gray regions GLA1 to GLA10 and the plurality of second gray regions GLB1 to GLB10 in each of the first image region and the second image region. Therefore, it becomes easy to estimate whether a light environment includes oblique light, based on whether a difference in brightness of gray images adjacent to each other is constant. Therefore, it becomes possible to easily acquire an appropriate skin image by photographing using light of a specific wavelength.

According to the present example embodiment, the plurality of first skin reflection regions SA1 to SA4 and the plurality of second skin reflection regions SB1 to SB4 are formed between the plurality of first gray regions GLA1 to GLA10, and the plurality of second gray regions GLB1 to GLB10.

This enables, by using the one test chart TC3, to evaluate and adjust a gradation of the camera 101, and evaluate or decide whether to be able to acquire a good image of a skin of a person. Therefore, it becomes possible to acquire an appropriate skin image by photographing using light of a specific wavelength more easily than a case, for example, where an individual test chart is used for gradation of the camera 101 and for evaluation of an image of a skin of a person.

According to the present example embodiment, the test chart TC3 includes the plurality of first gray regions GLA1 to GLA10, and the plurality of second gray regions GLB1 to GLB10. The plurality of first gray regions GLA1 to GLA10 are formed on the sheet member ST, and indicate depth different from one another. The plurality of second gray regions GLB1 to GLB10 are formed on the sheet member ST, and indicate depth associated with depth indicated by each of the plurality of first gray regions GLA1 to GLA10. The plurality of first gray regions GLA1 to GLA10 are arranged in order of depth in such a way that the depth indicated by each thereof changes with a certain difference. A first gray region and a second gray region indicating associated depth are formed at a symmetric position with respect to the common point C.

Further, in a case where a gray image group included in at least one of the first image region and the second image region among the plurality of gray images GLAim1 to GLAim10 and GLBim1 to GLBim10 does not have brightness according to associated depth of the plurality of first gray regions GLA1 to GLA10 and the plurality of second gray regions GLB1 to GLB10, the estimation unit 513 estimates that a light environment includes oblique light. The plurality of gray images GLAim1 to GLAim10 and GLBim1 to GLBim10 indicate, in a chart image, each of the plurality of first gray regions GLA1 to GLA10 and the plurality of second gray regions GLB1 to GLB10.

This enables to recognize whether a light environment in a photographing place includes oblique light, and handle in such a way that the light environment becomes appropriate, in a case where the light environment in the photographing place includes oblique light. Therefore, it becomes possible to acquire an appropriate skin image by photographing using light of a specific wavelength.

Each of the first image region and the second image region includes an image in a darkest gray region and an image in a lightest gray region in the plurality of first gray regions GLA1 to GLA10 and the plurality of second gray regions GLB1 to GLB10.

This enables to estimate whether a light environment includes oblique light, and handle in such a way that the light environment becomes appropriate, in a case where the light environment in the photographing place includes oblique light. Therefore, it becomes possible to acquire an appropriate skin image by photographing using light of a specific wavelength.

In the foregoing, example embodiments and modification examples according to the present disclosure have been described with reference to the drawings, however, these are examples of the present disclosure, and various configurations other than the above can also be adopted.

Further, in a plurality of flowcharts used in the above description, a plurality of processes (pieces of processing) are described in order, however, the order of execution of processes to be performed in each example embodiment is not limited to the order of description. In each example embodiment, the illustrated order of processes can be changed within a range that does not adversely affect a content. Further, the above-described example embodiments and modification examples can be combined, as far as contents do not conflict with each other.

A part or all of the above-described example embodiments may also be described as the following supplementary notes, but is not limited to the following.
1. A test chart including:
   a sheet member; and
   a plurality of first skin reflection regions formed on the sheet member, wherein
   each of the plurality of first skin reflection regions indicates a reflectance of a skin with respect to first wavelength light.
2. The test chart according to supplementary note 1, further including
   a plurality of second skin reflection regions formed on the sheet member, wherein
   each of the plurality of second skin reflection regions indicates a reflectance of a skin with respect to second wavelength light.
3. The test chart according to supplementary note 2, wherein
   the plurality of first skin reflection regions includes a first maximum region indicating a maximum reflectance of a skin with respect to the first wavelength light, and a first minimum region indicating a minimum reflectance of a skin with respect to the first wavelength light, and
   the plurality of second skin reflection regions includes a second maximum region indicating a maximum reflectance of a skin with respect to the second wavelength light, and a second minimum region indicating a minimum reflectance of a skin with respect to the second wavelength light.
4. The test chart according to supplementary note 2 or 3, wherein
   each beam of the first wavelength light and the second wavelength light is near infrared light.
5. The test chart according to any one of supplementary notes 2 to 4, further including
   a plurality of first gray regions that are formed side by side on the sheet member, and indicate depth different from one another.
6. The test chart according to supplementary note 5, further including
   a plurality of second gray regions that are formed on the sheet member, and indicate depth associated with depth indicated by each of the plurality of first gray regions, wherein
   the plurality of first gray regions are arranged in order of depth in such a way that depth indicated by each of the plurality of first gray regions changes with a certain difference, and
   the first gray region and the second gray region indicating associated depth are formed at a symmetric position with respect to a common point.
7. The test chart according to supplementary note 6, wherein
   the plurality of first skin reflection regions and the plurality of second skin reflection regions are formed between the plurality of first gray regions and the plurality of second gray regions.
8. An image analysis apparatus including:
   an image acquisition unit that acquires a chart image to be acquired by photographing a test chart; and
   an analysis unit that analyzes the chart image, wherein
   the test chart includes
   a sheet member, and
   a plurality of first skin reflection regions formed on the sheet member, and
   each of the plurality of first skin reflection regions indicates a reflectance of a skin with respect to first wavelength light.
9. The image analysis apparatus according to supplementary note 8, further including
   an adjustment unit that adjusts a predetermined target device, based on an analysis result of the chart image, wherein
   the analysis unit derives brightness of a plurality of first skin reflection images each indicating the plurality of first skin reflection regions, and
   the adjustment unit adjusts a target device in such a way that brightness of the plurality of first skin reflection images becomes equal to or less than brightness of a first maximum image indicating a first maximum region, and becomes equal to or more than brightness of a first minimum image indicating a first minimum region.
10. The image analysis apparatus according to supplementary note 8, further including
   an adjustment unit that adjusts a predetermined device, based on a result acquired by analyzing the chart image, wherein
   the analysis unit includes
   a region determination unit that determines a first image region and a second image region in the chart image, based on a predetermined region determination condition, and
   an estimation unit that estimates a light environment in a photographing place of the test chart, based on each of the first image region and the second image region, and
   the adjustment unit adjusts the target device, based on the estimated light environment.
11. The image analysis apparatus according to supplementary note 10, wherein
   the region determination condition is defined in associated with at least one of an illumination provided for photographing, a position of each of one or a plurality of illumination devices provided in the photographing place, and ambient light in the photographing place.
12. The image analysis apparatus according to supplementary note 10 or 11, wherein
   the estimation unit estimates whether each of a first light environment associated with the first image region, and a second light environment associated with the second image region is good in light of a predetermined decision criterion, and,
   in a case where it is estimated that one light environment between the first light environment and the second light environment is good, and it is estimated that other light environment is not good, the adjustment unit applies an operation condition of the target device associated with the one light environment to an operation condition of the target device associated with the other light environment.
13. The image analysis apparatus according to supplementary note 10 or 11, wherein
   the estimation unit estimates whether each of a first light environment associated with the first image region, and a second light environment associated with the second image region is good in light of a predetermined decision criterion, and,
   in a case where it is estimated that one light environment between the first light environment and the second light environment is good, and it is estimated that other light environment is not good, the adjustment unit adjusts the target device in such a way as to make the other light environment close to the one light environment.
14. The image analysis apparatus according to supplementary note 10, wherein
   the test chart includes
   a plurality of first gray regions that are formed on the sheet member, and indicate depth different from one another, and
   a plurality of second gray regions that are formed on the sheet member, and indicate depth associated with depth indicated by each of the plurality of first gray regions,
   the plurality of first gray regions are arranged in order of depth in such a way that the depth indicated by each of the plurality of first gray regions changes with a certain difference,
   the first gray region and the second gray region indicating associated depth are formed at a symmetric position with respect to a common point, and,
   in a case where a gray image group included in at least one of the first image region and the second image region among a plurality of gray images indicating, in the chart image, each of the plurality of first gray regions and the plurality of second gray regions does not have brightness according to associated depth of the plurality of first gray regions and the plurality of second gray regions, the estimation unit estimates that the light environment includes oblique light.
15. The image analysis apparatus according to supplementary note 14, wherein
   each of the first image region and the second image region includes an image in a darkest gray region and an image in a lightest gray region in the plurality of first gray regions and the plurality of second gray regions.
16. The image analysis apparatus according to any one of supplementary notes 8 to 15, further including
   a display control unit that generates a display image in which an analysis result of the chart image is superimposed on the chart image, and causes a display unit to display the generated display image.
17. An image analysis system including:
   a test chart;
   a camera that photographs the test chart and generates a chart image;
   a first light that irradiates first wavelength light to the test chart; and
   an image analysis apparatus, wherein
   the test chart includes
   a sheet member, and
   a plurality of first skin reflection regions formed on the sheet member,
   each of the plurality of first skin reflection regions indicates a reflectance of a skin with respect to the first wavelength light, and
   the image analysis apparatus includes
   an image acquisition unit that acquires a chart image to be acquired by photographing the test chart, and
   an analysis unit that analyzes the chart image.
18. An image analysis method including,
   by one or more computers:
   acquiring a chart image to be acquired by photographing a test chart; and
   analyzing the chart image, wherein
   the test chart includes
   a sheet member, and
   a plurality of first skin reflection regions formed on the sheet member, and
   each of the plurality of first skin reflection regions indicates a reflectance of a skin with respect to first wavelength light.
19. A storage medium storing a program for causing a computer to execute:
   acquiring a chart image to be acquired by photographing a test chart; and
   analyzing the chart image, wherein
   the test chart includes
   a sheet member, and
   a plurality of first skin reflection regions formed on the sheet member, and
   each of the plurality of first skin reflection regions indicates a reflectance of a skin with respect to first wavelength light.

### REFERENCE SIGNS LIST

100, 200, 300, 400, 500 Image analysis system
101 Camera
102 First light
103, 303, 403, 503 Image analysis apparatus
104 Image acquisition unit
105, 305, 405, 505 Analysis unit
106 Display unit
107 Display control unit
108 Brightness acquisition unit
109 Decision unit
111a to 111d, 211a to 211d Display image
202 Second light
311, 411 Adjustment unit
412 Region determination unit
413, 513 Estimation unit
TC1, TC2, TC3 Test chart
ST Sheet member
SA1 to SA4 First skin reflection region
SB1 to SB4 Second skin reflection region
SAim1 to SAim4 First skin reflection image
SBim1 to SBim4 Second skin reflection image
GLA1 to GLA10 First gray region
GLB1 to GLB10 Second gray region
GLAim1 to GLAim10 First gray image
GLBim1 to GLBim10 Second gray image

What is claimed is:

1. A test chart comprising:
a sheet member; and
a plurality of first skin reflection regions formed on the sheet member, wherein
each of the plurality of first skin reflection regions indicates a reflectance of a skin with respect to first wavelength light.

2. The test chart according to claim 1, further comprising
a plurality of second skin reflection regions formed on the sheet member, wherein
each of the plurality of second skin reflection regions indicates a reflectance of a skin with respect to second wavelength light.

3. The test chart according to claim 2, wherein
the plurality of first skin reflection regions includes a first maximum region indicating a maximum reflectance of a skin with respect to the first wavelength light, and a first minimum region indicating a minimum reflectance of a skin with respect to the first wavelength light, and
the plurality of second skin reflection regions includes a second maximum region indicating a maximum reflectance of a skin with respect to the second wavelength light, and a second minimum region indicating a minimum reflectance of a skin with respect to the second wavelength light.

4. The test chart according to claim 2, wherein
each beam of the first wavelength light and the second wavelength light is near infrared light.

5. The test chart according to claim 2, further comprising
a plurality of first gray regions that are formed side by side on the sheet member, and indicate depth different from one another.

6. The test chart according to claim 5, further comprising
a plurality of second gray regions that are formed on the sheet member, and indicate depth associated with depth indicated by each of the plurality of first gray regions, wherein
the plurality of first gray regions are arranged in order of depth in such a way that the depth indicated by each of the plurality of first gray regions changes with a certain difference, and
the first gray region and the second gray region indicating associated depth are formed at a symmetric position with respect to a common point.

7. The test chart according to claim 6, wherein
the plurality of first skin reflection regions and the plurality of second skin reflection regions are formed between the plurality of first gray regions and the plurality of second gray regions.

8. An image analysis apparatus comprising:
at least one memory configured to store instructions; and
at least one processor configured to execute the instructions to:
acquire a chart image to be acquired by photographing a test chart; and
analyze the chart image, wherein
the test chart includes
a sheet member, and
a plurality of first skin reflection regions formed on the sheet member, and
each of the plurality of first skin reflection regions indicates a reflectance of a skin with respect to first wavelength light.

9. The image analysis apparatus according to claim 8, wherein
the at least one processor configured further to execute the instructions to
adjust a predetermined target device, based on an analysis result of the chart image, wherein
analyzing the chart image includes deriving brightness of a plurality of first skin reflection images each indicating the plurality of first skin reflection regions, and
the target device is adjusted in such a way that brightness of the plurality of first skin reflection images becomes equal to or less than brightness of a first maximum image indicating a first maximum region, and becomes equal to or more than brightness of a first minimum image indicating a first minimum region.

10. The image analysis apparatus according to claim 8, wherein
the at least one processor configured further to execute the instructions to
adjust a predetermined device, based on a result acquired by analyzing the chart image, wherein
analyzing the chart image includes
determining a first image region and a second image region in the chart image, based on a predetermined region determination condition, and
estimating a light environment in a photographing place of the test chart, based on each of the first image region and the second image region, and
the target device is adjusted, based on the estimated light environment.

11. The image analysis apparatus according to claim 10, wherein
the region determination condition is defined in associated with at least one of an illumination provided for photographing, a position of each of one or a plurality of illumination devices provided in the photographing place, and ambient light in the photographing place.

12. The image analysis apparatus according to claim 10, wherein
estimating the light environment includes estimating whether each of a first light environment associated with the first image region, and a second light environment associated with the second image region is good in light of a predetermined decision criterion, and,
adjusting the target device includes, in a case where it is estimated that one light environment between the first light environment and the second light environment is good, and it is estimated that other light environment is not good, applying an operation condition of the target device associated with the one light environment to an operation condition of the target device associated with the other light environment.

13. The image analysis apparatus according to claim 10, wherein
estimating the light environment includes estimating whether each of a first light environment associated with the first image region, and a second light environment associated with the second image region is good in light of a predetermined decision criterion, and,
adjusting the target device includes, in a case where it is estimated that one light environment between the first light environment and the second light environment is good, and it is estimated that other light environment is not good, adjusting the target device in such a way as to make the other light environment close to the one light environment.

14. The image analysis apparatus according to claim 10, wherein
the test chart includes
a plurality of first gray regions that are formed on the sheet member, and indicate depth different from one another, and
a plurality of second gray regions that are formed on the sheet member, and indicate depth associated with depth indicated by each of the plurality of first gray regions,
the plurality of first gray regions are arranged in order of depth in such a way that the depth indicated by each of the plurality of first gray regions changes with a certain difference,
the first gray region and the second gray region indicating associated depth are formed at a symmetric position with respect to a common point, and,
estimating the light environment includes, in a case where a gray image group included in at least one of the first image region and the second image region among a plurality of gray images indicating, in the chart image, each of the plurality of first gray regions and the plurality of second gray regions does not have brightness according to associated depth of the plurality of first gray regions and the plurality of second gray regions, estimating that the light environment includes oblique light.

15. The image analysis apparatus according to claim 14, wherein
each of the first image region and the second image region includes an image in a darkest gray region and an image in a lightest gray region in the plurality of first gray regions and the plurality of second gray regions.

16. The image analysis apparatus according to claim 8, wherein
the at least one processor configured further to execute the instructions to
generate a display image in which an analysis result of the chart image is superimposed on the chart image, and cause a display to display the generated display image.

17. An image analysis system comprising:
a test chart according to claim;
a camera that photographs the test chart and generates a chart image;
a first light that irradiates first wavelength light to the test chart; and
an image analysis apparatus according to claim 8.

18. An image analysis method comprising,
by one or more computers:
acquiring a chart image to be acquired by photographing a test chart according to claim; and
analyzing the chart image.

19. A non-transitory computer readable medium storing a program for causing a computer to execute:
acquiring a chart image to be acquired by photographing a test chart according to claim; and
analyzing the chart image.
